(19) United States
(12) Patent Application Publication
(10) Pub. No.: US 2010/0305201 A1
(43) Pub. Date: Dec. 2, 2010

(54) Method of Treating a Tumor and Biodistribution of a Drug Delivered by Worm-Like Filomicelles

(75) Inventors: Dennis E. Discher, Philadelphia, PA (US); Shenshen Cai, Morgan Town, WV (US); Yan Geng, Langhorne, PA (US); Paul Dalhaimer, Sheboygan, WI (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: The Trustees of the University of Pennsylvania

(21) Appl. No.: 12/514,988

(22) PCT Filed: Nov. 14, 2007
(86) PCT No.: PCT/US2007/023777

§ 371 Date: Aug. 20, 2010

Related U.S. Application Data
(60) Provisional application No. 60/858,861, filed on Nov. 14, 2006.

(51) Int. Cl. A61K 31/337; C12N 5/02; A61P 35/00

(52) U.S. Cl. 514/449; 435/375

(57) ABSTRACT

Provided are filomicelle nanocarrier systems for the controlled transport and bioselective delivery of encapsulatable, cytotoxic active agents contained therein, particularly anticancer agents. Further provided are methods for controlling destabilization of the filomicelle membrane and the resulting hydrolysis-triggered, controlled release of the active agent(s) encapsulated therein by controlling the blend ratio (mol %) of hydrolysable PEO-block copolymer of the hydrophilic component(s) and of the more hydrophobic PEO-block copolymer component(s), wherein bioselective release of the encapsulated cytotoxic agents is distributed intracellularly, and wherein lowered dosage of the drug was delivered to the non-tumor organs. Thus, the filomicelle system offers enhanced tumor-selective biodistribution of a drug, and a reduced toxicity of the encapsulated drug to other organs.

### CROSS REFERENCE TO RELATED APPLICATIONS

This application claims priority to U.S. Provisional Application No. 60/858,861 filed Nov. 14, 2006, which is incorporated herein in its entirety.

### GOVERNMENT INTEREST

This invention was supported in part by Grant No. R21 from the National Institutes of Health. Accordingly, the Government may have certain rights in this invention.

### FIELD OF THE INVENTION

The present invention relates to filomicelle nanocarrier and their ability to deliver active agents, including drugs, specifically anticancer drugs, in vivo. In particular drug delivery is shown to be specific to tumors, as compared to other tissues in the patient, permitting lower dosage, more specific drugs to be delivered to cancer patients.

### BACKGROUND OF THE INVENTION

Parenteral delivery of chemotherapeutics is a cornerstone of clinical cancer treatment, but many drugs are hydrophobic and require a solubilizing carrier. Such systems must load and stably retain anticancer drugs and must also have a means to release drugs into cells. To meet this need, drug delivery is one of the most promising biomedical applications of nanotechnology, and several nanocarrier formulations, such as Doxil and Abraxane, have already been approved for clinical use in cancer chemotherapy (Ferrari, Nat. Rev. Cancer 5:161-171 (2005)). A good nanocarrier is one that circulates in the blood for long periods of time and delivers the drugs with minimal side effects (Duncan, Nat. Rev. Drug Discov. 2:347-360 (2003)).

Unless the nanoparticles have entered the cells in vivo, they are cleared in the first pass through the microvasculature of various bodily organs. In contrast, nanovehicles, such as viruses, liposomes or quantum dots, have been widely applied as gene, drug or dye carriers because they tend to circulate in vivo for a few hours, or perhaps a day (in rodents), and because they can enter cells. Accordingly, a number of popular nanocarriers based on liposomes (Gabizon et al., J. Control. Release 63:275-279 (1998) and polymeric micelles (Aliabadi et al., Expert. Opin. Drug Deliv. 3:139-162 (2006); Nishiyama et al., Pharmacol. Therapeut. 112:630-648 (2006)) can effectively protect and deliver poorly soluble drugs. Such nanocarriers are typically between 100 and 200 nm in diameter, are spherical in shape, and are modified with poly(ethylene glycol) (PEG) to improve circulation in the bloodstream. Although PEG modification is an effective strategy, it cannot prolong the circulation of such nanocarriers beyond 48 hours, and it is believed that nanocarriers larger than 200 nm are cleared even more quickly.

Since the first reported use of spherical micelles as drug vehicles in the late 1980s, block copolymer assemblies have gained increasing popularity in the field of drug delivery. To date, several spherical micellar formulations of anticancer agents are in clinical trials. However, in 2002, Discher and co-workers pioneered a new field of block copolymer assemblies (Discher et al., Science 297:967-973 (2002)), creating polymer vehicles (“polymersomes”) and cylindrical, worm- or filovirus-like micelles (“filomicelles”), suggesting their functions as nanocarriers and chemical reactors, and also as models to study biological systems.

The chemical design of block copolymers brings several advantages because the size, stability, drug loading and release efficiencies can be controlled by simply varying the ratio of the hydrophobic block in the polymer composition or by changing features, such as pH, in the surrounding environment. Such block copolymers, comprising of two or more different polymers, can spontaneously assemble into spherical, cylindrical, lamellar or vesicular shapes in selective solvents (Photos et al., J. Control. Release 90:323-334 (2003); Dalhaimer et al., Biomacromol. 5:1714-1719 (2004); Geng et al., J. Phys. Chem. B Condens. Matter Mater. Surf. Interfaces Biophys. 109: 3772-3779 (2005A); Geng et al., J. Am. Chem. Soc. 127:12780-12781 (2005B); Vijayanand et al., J. Polym. Sci. Part B—Polymer Phys. 44:3431-3433 (2006)). Studies have characterized the worm-like filomicelles and initial examined the potential of those micelles in drug delivery Dalhaimer et al., supra, 2004; Kim et al., Nanotechnol. 16:S484-S491 (2005); Cai et al., Pharm. Res. (2007) in press; Geng et al., Nature Nanotechnol. 2:249-255 (2007).

Paclitaxel (TAX) is a clinically prevalent anti-cancer agent that has been demonstrated to be effective in treatments on many types of solid tumors (Kim et al., Clin. Canc. Res. 10:3708-3716 (2004); Han et al., Acta Pharmacolog. Sinica 27:747-753 (2006); Tong et al., Rapid Communic. in Mass Spectrometry 20:1905-1912 (2006)). It has remained the primary chemotherapeutic agent used in non-small cell lung cancer (NSCLC) therapy so far. However, the severe side effects of TAX itself, as an anticancer chemotherapeutic to other normal organs (Blackhall et al., Current Opin. 4:71-84 (2005); Gligorovand et al., Oncologist 9:3-8 (2004)), also limits its high dose administration. Moreover, due to its poor solubility in aqueous solution, TAX at 0.3 mg/ml (25° C.) (Soga et al., J. Control. Release 103:341-353 (2005)), or 3-4 mg/ml at 37° C. (Liggins et al., J. Pharm. Sci. 86:1458-1463 (1997) covalent modifications to a variety of both formulation and delivery systems have been studied in an attempt to increase in vivo absorption and to enhance solubility, stability, loading capacity in delivery systems, and in vivo antitumor efficacy (Dhanikula et al., Curr. Drug Deliv. 2:35-44 (2005)).

These formulation methods include, but are not limited to, the usage of amphiphilic polymeric micelles (Kim et al., Polymers for Adv. Technol. 10:647-654 (1999); Park et al., J. Control. Release 109:158-168 (2005); Zhang et al., Cancer Chemotherapy & Pharmacol. 40:81-86 (1997); Zhang et al., Anti-Cancer Drugs 8:696-701 (1997)), vesicles, liposomes (Fetterlyand et al., AAPS Pharm. Sci. 5:E32 (2003)), nanospheres (Kimand et al., Biomaterials 22:1697-1704 (2001); Kim et al., Biomaterials 24:55-63 (2003)), nanoparticles (Hamaguchi et al., Brit. J. Canc. 92:1240-1246 (2005); Moand et al., J. Control. Release 107:30-42 (2005); Xie et al., Pharmaceut. Res. 22:2079-2090 (2005)), microspheres (Harper et al., Clin. Canc. Res. 5:4242-4248 (1999)), oil emulsification systems (Veltkamp et al., Brit. J. Canc. 95:729-734 (2006)), films (Dhanikula et al., AAPS J. 6(3):Article 27 (2004)), and water-soluble prodrugs (Niethammer et al., Bioconjugate Chem. 12:414-420 (2001); Zou et al., Clin. Cancer Res. 10:7382-7391 (2004)).

As the most common emulsifying agent used in the clinic to solubilize TAX, Cremophor® EL is a complex, viscous mixture composed primarily of hydrophobic glycerolpolyoxyethylene ricinoleates, various fatty acid esters, and 250% ethanol (Cheon Lee et al., J. Control. Release 89:437-446 (2003); Meyer et al., Electrophoresis 23:1053-1062 (2002);). Yet, clinical problems associated with Cremophor EL include low drug stability after dilution in aqueous medium (Moand et al., supra, 2005) and severe, dose-limiting, toxic side effects, such as hypersensitivity and cardiotoxicity (Kim et al., J. Control. Release 72:191-202 (2001); Fetterly et al., Biopharm. Drug Dispos. 22:251-61 (2001); Xie et al., supra, 2005; Liu et al., Curr. Pharmaceut. Design 12:4685-4701 (2006)). TAX loaded into liposome membranes has effectively reduced both the toxicity typical of TAX emulsions Dorr, Ann. Pharmacother. 28:11-14 (1994) and TAX's neurotoxicity (Park, Breast Cancer Res. 4:95-99 (2002)), but high concentrations of TAX destabilize thin lipid membranes (Immordino et al., J. Control. Research 91:417-429 (2003)).

This has motivated the development of copolymer micelles with cores tailored to hydrophobic drugs (Iijima et al., Macromolecules 32:1140-1146 (1999); Shuai et al., Bioconjugate Chem. 15:441-448 (2004)). Likewise, liposomal DOX and polymer-based DOX carriers limit rapid excretion of free drug (Sengupta et al., Nature 436:568-572 (2005); Bae et al., Bioconjugate Chem. 16:122-130 (2005); Omayra et al., Bioconjugate Chem. 13:453-461 (2002)), as well as cardiotoxicity (Kluza et al., Oncogene epub 1-13 (2004)). But, liposomes often prove leaky and lipid PEG-ylation limits drug release at the tumor (Hong et al., Clin. Cancer Res. 5:3645-3652 (1999)). Consequently, although polymeric micelles are still highly recognized candidates for TAX delivery because of their sizable hydrophobic cores and good stabilities, but not all are biocompatible. Thus, there remains a need for safer and more effective TAX delivery systems.

Amphiphilic diblock copolymers generally self-assemble in dilute aqueous solution into three basic morphologies: spherical micelles, worm-like micelles, and vesicles. Spherical micelles form spontaneously when the hydrophilic, corona block such as poly(ethylene oxide) (PEO) is the largest block by mass, and these have now been widely studied in bio-application. Following parental administration, such spheres delay clearance by macrophages of the liver and spleen due to the hydrated corona and also—it has been thought—due to their small size (Gaucher et al., J. Control. Release 109:169-188 (2005)). Escape from clearance in principle allows accumulation in tumors, and use of copolymers that are degradable (Yoshii et al., Radiat. Phys. Chem. 57:417-420 (2000); Liggins et al., Inflamm. Res. 53:363-372 (2004)) or sensitive to temperature or pH can provide mechanisms for controlled drug release (Soga et al, supra, 2005; Liu et al., supra, 2005; Gao et al., J. Drug Target. 13:391-397 (2005)). By decreasing the weight fraction of the PEO block to just less than 250%, hydration and swelling of the corona imparts just enough curvature to the copolymer assembly that worm-like micelles that are microns in length and similar in diameter to the spheres are the predominant morphology for a variety of diblock copolymers (Geng et al., supra, 2005B; Dalhaimer et al., Macromolecules 36:6873-6877 (2003)). Drugs, such as TAX and various hydrophobic dyes have now been loaded into these novel carriers (Dalhaimer et al., supra, 2003; Geng et al., supra, 2005A & B; Kim et al., supra, 2005), and worm-like micelles persist for up to 1 week in vivo in the blood circulation, which appears to be longer than any other synthetic particle, including stealthy vesicles bearing the same length of PEO chains (Geng et al., Nat. Nanotech, 2007 in press).

Thus, it has been demonstrated that worm-like filomicelles have similar diameters and hydrophobic cores to spheres despite the extended contour length, and they can also readily encapsulate hydrophobic molecules, such as hydrophobic dyes and TAX. More importantly, worm-like filomicelles have proven to have several advantages over spherical micelles including up to 2-fold higher loading capacity and efficiency (Cai et al., supra, 2007), and longer in vivo blood circulation time up to 3-4 days (Geng et al., supra, 2007). Consequently, there remains a need in the art for a reliable, nontoxic method for delivering hydrophobic drugs by tumor-specific delivery in vivo to achieve consequent therapeutic effects, while at the same time avoiding the deleterious effects of high-dosages of the often toxic compositions.

### SUMMARY OF THE INVENTION

The side effects of anticancer drugs are always a substantial problem remaining in cancer chemotherapies. As chemotherapeutics kill the tumor cells, they also impose toxicity to other critical major organs, which significantly limit the dose that can be applied to patients. However, the morphology of filomicelles is shown herein to offer a better TAX carrier system than traditional spherical micelles, not only in their ability to enhance the maximum tolerated dose thereby allowing higher doses to be administered to achieve better tumor inhibition prognosis, but also in their better tumor selectivity with far less toxicity to other non-tumor organs. Further, the present invention provides a reliable, nontoxic method for delivering hydrophobic drugs by tumor-specific delivery in vivo to reduce the size of the tumors by the use of reduced dosages.

It is an object of this invention to provide biocompatible nanocarriers for the delivery of hydrophobic compositions, such as drugs, particularly anticancer or antitumor drugs, to a patient in need thereof, and to execute the consequent therapeutic effects in vivo. Particularly useful for this purpose is a delivery system using highly flexible, worm-like micelles, referred to as “filomicelles,” that are nanometers in cross-section and spontaneously assemble from block copolymers to stable assemblies that are microns in length, which can also be sonicated to generate kinetically stable spherical micelles of the same copolymer that encapsulate the hydrophobic active agent compositions.

Following intravenous administration of TAX-loaded filomicelles into mice, when compared to delivery by other systems, TAX release was found to be enhanced from a filomicelle delivery system at lower pH, which is favorable as cancerous tissues are generally associated with acidic environment. Moreover, there was significantly less cytotoxicity and greater potency in delivering TAX to human lung carcinoma A549 cells by the filomicelle delivery system, as compared to Cremophor® EL. Thus, it is a further object of the invention to provide a higher administered dose of a drug, such as TAX, into tumor-bearing mice with guaranteed safety, while at the same time achieving enhanced tumor shrinkage.

Cell apoptosis analyses further demonstrate that worm-like filomicelles induced cell apoptosis more significantly in tumor than in other non-tumor organs, which is consistent with the tumor shrinkage experiment using different TAX formulations. In addition, biodistribution studies confirmed the enhanced tumor-selective distribution of TAX delivered by worm-like filomicelles. Thus, PEO-PCL (OCL) based worm-like filomicelles offer novel tumor-specific and/or anticancer drug delivery systems using enhanced drug toleration doses and better tumor-selective delivery patterns in vivo than traditional spherical micelles, thus offer a promising new system for anti-cancer drug delivery.

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE DRAWINGS

The foregoing summary, as well as the following detailed description of the invention, will be better understood when read in conjunction with the appended drawings. It should be understood, however, that the invention is not limited to the precise arrangements and instrumentalities shown.

FIGS. 1A and 1B are schematic diagrams. FIG. 1A illustrates the preparation of worm-like filomicelles and of spherical micelles, each from poly(ethylene oxide)-b-poly(ε-caprolactone) (PEO-PCL, denoted OCL). FIG. 1B illustrates in vivo TAX delivery by spherical vesicles and by worm-like filomicelles, showing that worm-like filomicelles accumulate more in tumor over other non-tumor organs due to the tumor-featured enhanced permeation and retention (EPR) effect. FIG. 2 shows a size and diffusion analysis of OCL3 micelles as measured by DLS.

FIGS. 3A-C3 show the effect of an HPLC-UV assay for paclitaxel (TAX) encapsulation. FIG. 3A is a plot of HPLC-UV-determined area-under-curve (AUC) ratio between TAX and docetaxel vs TAX concentration. Inset shows the HPLC-UVspectrum of docetaxel (as internal standard) and paclitaxel (TAX). FIG. 3B TAX loading capacity (upper panel, defined as mg TAX loaded per milligram (mg) micelle) and solubilization (lower panel, defined as mg TAX loaded per milliliter (ml) aqueous solution) with OCL3 micelles when total added TAX:OCL3 micelle (w/w)=1:5. FIG. 3C shows the ratio between TAX loading capacity with OCL3 worms and spheres at different conditions. Total added TAX:OCL3 micelle (w/w) was fixed at 1:5 while OCL3 concentration varied when added TAX concentration≦2 mg/ml, and OCL3 micelle concentration was fixed at 10% (w/v) when added TAX concentration≧2 mg/ml.

FIGS. 4A-4C show the effect of extrusion, TAX loading, freeze/thaw and storage on micelle morphology and leakage of encapsulated drug. FIG. 4A shows the effect of extrusion, TAX loading and 1 month of storage on the effective size of the filomicelle as determined by DLS. FIG. 4B shows the effect of 1 month storage at 4° and −20° C. on TAX leakage from the filomicelles. FIG. 4C shows the effect of multiple freeze/thaw cycles at −20° C. on TAX leakage from micelles.

FIGS. 5A-5D presents the results of a cytotoxicity study and IC50 evaluation of TAX-loaded micelles on A549 human lung carcinoma cells. FIG. 5A shows the extent of cytotoxicity of an excipient only (OCL3 spheres, worms, and Cremophor EL) on A549 cells. FIG. 5B shows a comparison of the excipient concentration at which 20% cells were killed (IC80). FIG. 5C shows the cytotoxicity of different TAX formulations on A549 cells. FIG. 5D shows a comparison of IC50 of different TAX formulations.

FIGS. 6A-6C show tumor inhibition by delivered TAX. FIG. 6A shows the result of tumor inhibition studies on lung carcinoma A549-bearing mice with multiple injections of: Controls (DPBS alone, empty OCL3 spherical or worm-like micelles), free TAX in DPBS, OCL3 spheres loaded with TAX at MTD (˜8 mg/kg) and OCL3 filomicelles loaded with TAX at MTD (˜16 mg/kg). Inset FIG. 6B shows MTD determination with TAX encapsulated OCL3 micelles in spherical or worm-like morphologies in nude mice. The tumor growth profile was determined by tumor area (A), which was monitored 24 hrs after each injection by measuring two orthogonal dimensions. The tumor growth-inhibition curves were fit by applying the exponential modeling equation (Equation 7). FIG. 6C shows body weight changes measured at 24 hrs after each injection during the 3-week experiment process, showing the mice were actually gaining, rather than losing weight for all experimental groups.

FIG. 7 shows that no body weight loss was observed in the test animals when administered the polymeric carriers alone (empty OCL3 micelles) having either spherical (Sph) or worm-like filamentous (FIL) morphologies in nude mice by monitoring body weight changes throughout the tested range over 24 hours.

FIGS. 8A and 8B show cell apoptosis evaluations of tumor and non-tumor major organs of the different multiple injection treatment groups after 22 days. FIG. 8A presents cell apoptosis index measured by ELISA and calculated as: Apoptosis index=Enrichment factor of TAX treatment animal groups/Enrichment factor of untreated animal groups. FIG. 8B presents the cell apoptosis index ratio between tumors and other non-tumor organs (p<0.05).

FIG. 9 is a plot of the relationship between relative tumor volume shrinkage vs. relative tumor apoptosis at day 22.

FIGS. 10A and 10B depict biodistribution data. FIG. 10A is a biodistribution assay of TAX in different organs after 24 hrs following a single I.V. injection, using RP-HPLC method. FIG. 10B plots normalized apoptosis vs. TAX distribution for tumor, liver and spleen, fit by y=mx+b, where m (tumor, liver, spleen)=(0.45, 0.24, 0.26), respectively.

FIG. 11 plots the release of TAX from OCL3 polymeric micelles DPBS buffer and in 1:1 DPBS:fetal bovine serum (FBS), showing that the addition of serum had no effect on the TAX release rate.

FIGS. 12A-12C show the entry of OS worm-like filomicelles into A549 cells. FIG. 12A shows PKH26 fluorescence dye labeled OS micelles accumulated to the cell membrane followed by intracellular uptake at different time points after dissociation into nano-sized spheres, scale bar=10 μm. FIG. 12B shows fluorescence intensity of cytoplasm increased with time, indicating the gradual cellular uptake of the OS micelles. FIG. 12C shows that the OS filomicelles remaining in the media decreased in length over time, suggesting their fragmentation.

FIGS. 13A-13C show the cellular entry of worm-like filomicelles. FIG. 13A is a calibration curve showing the linear relationship between the PKH26 fluorescence intensity and the length of the filomicelles, indicating that the dye was homogenously distributed along the worms and the fluorescence intensity is proportional to the size of the micelles. FIG. 13B shows enlarged A549 cells with uptake of OS micelles in a size of 100-300 nm (indicated by the star) as calculated from the calibration curve in FIG. 13A. Scale bar=5 μm. FIG. 13C diagrammatically shows the dissociation and endocytosis-assisted internalization of filomicelles into tumor cells.

FIGS. 14A and 14B show filomicelles mediate paclitaxel (TAX) delivery to rapidly growing tumor xenografts on nude mice. Controls were either saline or OCL3 filomicelles or TAX as free drug in ethanol at maximum tolerated dose or TAX loaded at two doses into the hydrophobic cores of filomicelles of two lengths. FIG. 14A shows apoptosis was measured at 1 week by quantitative imaging of TUNEL-stained tumor sections. FIG. 14B shows decreasing tumor size with increasing apoptosis following treatment. All data shows the average from four mice. The error bars=standard deviation.

### DESCRIPTION OF PREFERRED EMBODIMENTS OF THE INVENTION

Previous studies have shown that the amphiphilic diblock copolymer, poly(ethylene oxide)-b-poly(ε-caprolactone) (PEO-PCL) could form worm-like filomicelles and have advantages over spherical micelles made with the same copolymer, with substantially increased drug loading capacity and efficiency due to their cylindrical structures.

The present invention provides methods for controlled, tissue-specific, or sustained release from nano-scale carriers, known as “filomicelles,” for the delivery of hydrophobic drugs in vivo. When used to reduce the size of the tumors, this delivery system is beneficial from a clinical standpoint because patients can receive a single bolus injection instead of relying on long-term infusion, while at the same time avoiding the deleterious side-effect of high-dosages because the intended effect of the drug is focused on the cancer cells. Thus, the methods of the present invention rely upon the finding that synthetic nanodevices inspired from natural systems can have surprisingly interesting properties and behaviors that could change the design of drug-delivery vehicles, and can significantly improve the quality of life of patients receiving cancer chemotherapy.

“Nanotechnology” as used herein when the filomicelle operates as a “nanotube” or “nanodevice,” or transports an encapsulated active agent as a “nanocarrier,” refers broadly to a field of applied science and technology whose unifying theme is the control of matter on the atomic and molecular scale, normally 1 to 100 nanometers, and the fabrication of devices within that size range. It is a highly multidisciplinary field, drawing from fields such as applied physics, materials science, interface and colloid science, device physics, supramolecular chemistry, chemical engineering, mechanical engineering, and electrical engineering. Thus, nanotechnology can be seen as an extension of existing sciences into the nanoscale. “Nano-transforming” filomicelles, as the term is used in the present invention, refer to the nano-transforming assemblies comprising degradable polymeric materials with hydrolysable backbones. The degradable polyester, typically polylactide or polycaprolactone, as the hydrophobic block, can be connected to biocompatible polyethylene oxide (PEO) as the hydrophilic block. Degradation of the hydrolysable backbones results in changes in morphology of the vesicles as will be discussed below.

Rigid carbon nanotubes, when used for drug delivery, have in the past been cleared from the circulation in hours (Singh et al., Proc. Natl. Acad. Sci. USA 103:3357-3362 (2006)). Likewise, Discher and co-workers found that solid cylindrical micelles with a cross-linked core disappear in a similarly short amount of time. However, filamentous worm micelles, or filomicelles, are attractive nano-scale structures for drug delivery applications. When incubated with phagocytes (cells in the liver and spleen that remove foreign material) under flow conditions, the long filomicelles stretched out along the streamlines of blood flow and were less frequently captured and cleared by the cells, as compared to spherical vesicles. The shear forces on the long structures overwhelmed the phagocytosing cells that are responsible for clearance of foreign bodies in vivo, whereas smaller particles adhered to, and were more easily taken up by, the cells. Circulation times seem to modulate the ability of the structures to relax and/or fragment owing to flow or interactions with the cells.

Circulation times of nanocarriers are generally limited to hours (or up to one day) because of either rapid clearance by the mononuclear phagocytic system (MPS) of the liver and spleen, or by excretion. Clinical studies have shown that circulation times of spherical carriers are generally extended threefold in humans over rats, so circulation times for filomorphologies could approach one month in humans. As proposed for clinically used drug formulations of PEG-liposomes, long circulating filomicelles would increase the drug exposure to cancer cells and increase the time-integrated dose, commonly referred to in drug delivery as ‘the area under the curve.’ Additionally, the enhanced permeation and retention effect that allows small solutes and micelles to permeate the leaky blood vessels of a rapidly expanding tumor might also allow nanodiameter filomicelles to transport into the tumor stroma.

Persistent circulation of the nanocarriers has many practical applications because these vehicles can increase exposure of drugs to cancer cells. As will be described in greater detail below, when filomicelles were loaded with paclitaxel (TAX), a common hydrophobic anticancer drug, and injected into mice, it was found that an increase in filament length at a given drug dosage showed the same relative therapeutic effect as a similar increase in paclitaxel dosage. This is significant because for drugs that are effective, but also toxic to the patient at high doses, it means that much lower doses of the drug can be used, yet still achieve therapeutic effect.

The filomicelles (also referred to herein as “worm micelles,” “worm-like micelles,” and the like) can be conveniently constructed by the self-assembly in water or aqueous solution of high molecular weight, amphiphilic di-block copolymers (e.g., >1-4000 g/mol), according to known methods of the spontaneous, entropy-driven process of preparing a closed semi-permeable membrane. Thus, filomicelles, in contrast to early worms prepared from low molecular weight lipids and surfactants, offer stable, synthetic, non-living assemblies, even at body temperature (37° C.).

The preferred copolymers comprise a hydrophilic PEO (polyethyleneoxide) block and one of several hydrophobic blocks, such as an inert polyethylene or a biodegradable polylactone, that drive self-assembly of worm-like micelles, up to microns in length, in water and other aqueous media. The diblock or triblock copolymer amphiphiles that mimic the flexibility of various cytoskeletal filaments are described, for example, in U.S. Pat. No. 6,835,394, and in the pending patent applications related thereto, including U.S. Ser. No. 10/882,816, herein incorporated by reference.

“Polymers” are macromolecules comprising connected monomeric heterogeneous molecules. The monomeric units may be of a single type (homogeneous), or a variety of types (heterogeneous). The physical behavior of the polymer is dictated by several features, including the total molecular weight, the composition of the polymer (e.g., the relative concentrations of different monomers), the chemical identity of each monomeric unit and its interaction with a solvent, and the architecture of the polymer (whether it is single chain or branched chains). The physical behavior of the polymer is dictated by several features, including the total molecular weight, the composition of the polymer (e.g., the relative concentrations of different monomers), the chemical identity of each monomeric unit and its interaction with a solvent, and the architecture of the polymer (whether it is single chain or branched chains). For example, in polyethylene glycol (PEG), which is a polymer of ethylene oxide (EO), the chain lengths of which, when covalently attached to a phospholipid, optimize the circulation life of a liposome, is known to be in the approximate range of 34-114 covalently linked monomers (EO₃₄ to EO₁₁₄). The preferred copolymers comprise a hydrophilic PEO (polyethylene oxide) block and one of several hydrophobic blocks that drive self-assembly of the filomicelles, up to microns in length, in water and other aqueous media.

To form a stable membrane in water, the absolute minimum requisite molecular weight for an amphiphile must exceed that of methanol HOCH₃, which is undoubtedly the smallest canonical amphiphile, with one end polar (HO—) and the other end hydrophobic (—CH₃). Formation of a stable lamellar phase more precisely requires an amphiphile with a hydrophilic group whose projected area is approximately equal to the volume divided by the maximum dimension of the hydrophobic portion of the amphiphile (Israelachvili, in Intermolecular and Surface Forces, Pt3 (Academic Press, New York) 1995).

The assemblies embodied in the present invention comprise semi-permeable, thin-walled encapsulating membranes, tens of nanometers to tens of microns in length, made by self-assembly in various aqueous solutions of purely synthetic, amphiphilic molecules having an average molecular weight of many kilograms per mole. As a result, the filomicelles with or without encapsulated material are also referred to herein as “assemblies.” Such molecules are referred to as “super-amphiphiles” because of their large molecular weight in comparison to other amphiphiles, such as the phospholipids and cholesterol of eukaryotic cell membranes.

Synthetic amphiphiles having molecular weights less than a few kilodaltons, like natural amphiphiles, are pervasive as self-assembled, encapsulating membranes in water-based systems. These include complex fluids, soaps, lubricants, microemulsions consisting of oil droplets in water, as well as biomedical devices such as vesicles. “Complex fluids” are fluids that are made from molecules that interact and self-associate, conferring novel, rheological, optical, or mechanical properties on the fluid itself. Complex fluids are found throughout biological and chemical systems, and include materials such as biological membranes or biomembranes, polymer melts and blends, and liquid crystals. The self-association and ordering of the molecules within the fluid depends on the interaction between component parts of the molecules, relative to their interaction with solvent, if present.

An “encapsulating membrane,” as the term is used to refer to the filomicelles of the present invention, compartmentalize by being semi- or selectively permeable to solutes, either contained inside or maintained outside of the spatial volume delimited by the membrane. Thus, a filomicelle is an assembly formed in aqueous solution, which in most embodiments also contains an aqueous solution. However, the interior or exterior of the filomicelle could also be another fluid, such as oil or a gas. A “filomicelle,” as the term is used in the present invention, refers to the encapsulating membrane plus the space enclosed within the membrane.

The relevant class of super-amphiphilic molecules is represented by, but not limited to, block copolymers, e.g., hydrophilic polyethyleneoxide (PEO) linked to hydrophobic polyethylethylene (PEE), or polylactic acid (PLA). The PEO block of the polymer (which is the same as polyethylene-glycol; PEG) is widely known to make interfaces very biocompatible. Thus, the resulting worm micelles are amphiphilic aggregates poised in size between molecular scale spherical micelles and much larger lamellar structures, such as vesicles, and fluidity and hydrodynamics play important roles in their formation. The synthetic diversity of block copolymers provides the opportunity to make a wide variety of vesicles with material properties that greatly expand what is currently available from the spectrum of naturally occurring phospholipids.

An “amphiphilic” substance is one containing both polar (water-soluble) and hydrophobic (water-insoluble) groups, and polymers are macromolecules comprising connected monomeric units. The monomeric units may be of a single type (homogeneous), or a variety of types (heterogeneous). The physical behavior of the polymer is dictated by several features, including the total molecular weight, the composition of the polymer (e.g., the relative concentrations of different monomers), the chemical identity of each monomeric unit and its interaction with a solvent, and the architecture of the polymer (whether it is single chain or branched chains).

The most common lamellae-forming amphiphiles also have a hydrophilic volume fraction between 20 and 50%. Such molecules form, in aqueous solutions, bilayer membranes with hydrophobic cores never more than a few nanometers in thickness. The present invention relates to all super-amphiphilic molecules which have hydrophilic block fractions within the range of 20-50% by volume and which can form filomicelles. The ability of amphiphilic and super-amphiphilic molecules to self-assemble can be largely assessed, without undue experimentation, by suspending the synthetic super-amphiphile in aqueous solution and looking for lamellar and vesicular structures as judged by simple observation under any basic optical microscope or through the scattering of light.

The “lipid bilayer” comprises a double layer of phospholipid/diacyl chains, wherein the hydrophobic fatty acid tails of the phospholipids face each other and the hydrophilic polar heads of each layer face outward toward the aqueous solutions. Numerous receptors, steroids, transporters and the like are embedded within the bilayer of a typical cell. Thus, a “lipid vesicle” or “liposome,” is an inner space surrounded by a membrane comprising one or more phospholipids. However, unlike liposomes, a polymersome does not include lipids or phospholipids as its majority component. Consequently, filomicelles can be thermally, mechanically, and chemically distinct and, in particular, more durable and resilient than the most stable of lipid vesicles. In one exemplary implementation, filomicelles are neutral, nano-transforming particles.

A “membrane,” as the term is used in this invention, is a spatially distinct collection of molecules that defines a 2-dimensional surface in 3-dimensional space, and thus separates one space from another in at least a local sense. Such a membrane must also be semi-permeable to solutes, sub-microscopic in its thickness (d), and result from a process of self-assembly. It can have fluid or solid properties, depending on temperature and on the chemistry of the amphiphiles from which it is formed. At some temperatures, the membrane can be fluid (having a measurable viscosity), or it can be solid-like, with an elasticity and bending rigidity. The membrane can store energy through its mechanical deformation, or it can store electrical energy by maintaining a transmembrane potential. Under some conditions, membranes can adhere to each other and coalesce (fuse). Soluble amphiphiles can bind to, and intercalate within a membrane.

The preferred class of polymer selected to prepare the worm micelles of the present invention is the amphiphilic “block copolymer.” Block copolymers are polymers having at least two, tandem, interconnected regions of differing chemistry. Each region comprises a repeating sequence of monomers. Thus, a “diblock copolymer” comprises two such connected regions (A-B); a “triblock copolymer,” three (A-B-C), etc. Each region may have its own chemical identity and preferences for solvent. Thus, an enormous spectrum of block chemistries is theoretically possible, limited only by the acumen of the synthetic chemist.

A linear diblock copolymer of the form A-B can form a variety of different structures. In either pure solution (the melt) or diluted into a solvent, the relative preferences of the A and B blocks for each other, as well as the solvent (if present) will dictate the ordering of the polymer material, producing the numerous resulting structural phases. In the “melt” (pure polymer), a diblock copolymer may form complex structures as dictated by the interaction between the chemical identities in each segment and the molecular weight. The interaction between chemical groups in each block is given by the mixing parameter or Flory interaction parameter, which provides a measure of the energetic cost of placing a monomer of ‘A’ next to a monomer of ‘B.’ See, e.g., U.S. Pat. No. 6,835,394; U.S. Ser. No. 10/882,816 and Publ. US Pat. Appl. 2005/0180922 (herein incorporated by reference). Assembly of diblock copolymer amphiphiles into one of the worm micelles depends primarily on the weight fraction (w) of the hydrophilic block relative to the total copolymer molecular weight.

U.S. Ser. No. 10/913,660 and Publ. US Pat. Appl. 2006/0180922 (each of which is herein incorporated by reference), further provide methods for controlling the release of an encapsulated material from a worm micelle. For example, the worm-like micelles can be fragmented to sub-micron lengths, if desired, and they will flow through nanoporous matrices, including recognized models for brain tissue matrix. The synthetic micelle membrane can exchange material with the “bulk,” i.e., the solution surrounding the micelles. Each component in the bulk has a partition coefficient, meaning it has a certain probability of staying in the bulk, as well as a probability of remaining in the membrane. Conditions can be predetermined so that the worm micelles can be formed with a selected molecule, such as a drug or antigen, incorporated within the membrane, so that by controlling the partition coefficient, the molecule will be released into the bulk when the micelle arrives at a destination having a higher partition coefficient.

As disclosed in US Pat Pub. 2006/0165810, incorporated by reference, the ability to control and regulate the hydrolytic shortening (fragmentation) of worm micelles was demonstrated using worm micelles prepared from poly(ethylene oxide)-block-poly(ε-caprolactone) copolymers (PEO-PCL, also denoted OCL). This demonstrated that the rate of shrinkage of a worm-micelle can be controlled by the composition of the block copolymers, as well as by controlling stimuli, such as the pH and temperature of the environment in which the worm micelles are placed. By controlling the hydrolytic shortening of worm micelles, the delivery and release of materials encapsulated within the worm micelle is controlled. Adjustments of molecular weight, composition and polymerization of the micelle can be readily adapted to the size and viscosity of the selected drug by one of ordinary skill in the art using standard techniques, and the release of an encapsulated active agent can be controlled by the length of the worm, selectively persisting for hours to more than a month. Once the encapsulated active agent has been released and the worm has been fragmented, the fragments are then quickly removed from the patient's circulation.

In one embodiment, the exemplified filomicelles provide controlled release of encapsulated materials through a blend ratio (mol %) of hydrolysable PEO-block copolymer of the hydrophilic component(s) and of the more hydrophobic PEO-block copolymer component(s) of the filomicelle to produce amphiphilic high molecular weight PEO-based assemblies, wherein the PEO volume fraction (f_{EO}) and chain chemistry control encapsulant release kinetics from the copolymer vesicles and filomicelle carrier membrane destabilization. In an exemplary implementation, PEO filomicelles of the present invention have a diameter (thickness) in a range of approximately <50 nm to 200 nm, although the dimensions can vary depending on the encapsulated material.

The worm-like micelle assemblies of the present invention are advantageously stable in blood and in blood flow in vitro and in vivo. When compared with typical phospholipids in the prior art, having two acyl chains, temperature can affect the stability of the thin lamellar structures, in part, by determining the volume of the hydrophobic portion. In addition, the strength of the hydrophobic interaction, which drives self-assembly and is required to maintain membrane stability, is generally recognized as rapidly decreasing for temperatures above approximately 50° C. Consequently, as compared with the stable assemblies of the present invention, such vesicles generally are not able to retain their contents for any significant length of time under adverse conditions, such as boiling or freeze/thaw.

Because of the filomicelle's bilayer perselectivity, materials may be “encapsulated” into the aqueous interior or intercalated into the hydrophobic membrane core of the filomicelle of the present invention. In a preferred embodiment, phospholipid molecules have been shown to be incorporated within filomicelle membranes by the simple addition of the phospholipid molecules to the bulk. However, numerous technologies can be developed from such assemblies, owing to the numerous unique features of the bilayer membrane and the broad availability of super-amphiphiles, such as block copolymers. The present invention, therefore, provides filomicelles which encapsulate one or more “active agents,” which include, without limitation, active agent compositions, such as drugs, therapeutics, hormones, nutrients, proteins, salts and the like, including antisense molecules, ribozyme molecules or siRNA or RNAi molecules, or fragments thereof, forming a “loaded” or “encapsulated” filomicelle which is intended to transport an encapsulatable material (an “encapsulant”) to or from its immediately surrounding environment. As a result, by controlling the partition coefficient, the molecule is released into the bulk when the polymersome arrives at a destination having a higher partition coefficient.

Worm Flexibility and Delivery of Active Agent. The two types of worms—fluid or cross-linked—represent the two extremes at either end of a continuous stiffness scale that can be experimentally realized by blending saturated polyethylethylene (PEE) copolymer with the cross-linkable polybutadiene (PBD) copolymer. The two copolymers have already been shown (using membranes) to be fully miscible, and the PBD can be successfully reacted to give a range of stabilities and stiffnesses (see, e.g., U.S. Pat. No. 6,835,394). As shown, worm-like micelles can emulate the bending rigidity of various ubiquitous biopolymers, from intermediate filaments to microtubules, through selection of different sized copolymers and chemical fixation of unsaturated butadiene bonds.

As described in detail by the inventors in Publ. US Pat. Appl. 2005/0180922, visualization of the worm-like micelles can be achieved by fluorescence microscopy after incorporating fluorescent dyes into the micelle cores dyes, now a straight-forward process with hydrophobic dyes. Although the diameter (d) of the worm micelles is similar to the membrane thickness of polymersomes, the Brownian dynamics of worm micelles are highly pronounced in contrast to the membranes. Increasing the molecular weight of the copolymers increases both the diameter of the worm-like micelles (from about 10 to 40 nm or 60 nm) and their stiffness. Despite the fact that these are fluid assemblies, the stability of the worms is clear, however, and appears fully consistent with the forces that drives membrane formation and underlies the stability of the micelles. More importantly, in a flow field, such as in a patient's circulatory system, rather than under quiescent conditions, the fluid worms orient and stretch with DNA-like scaling, and respond in a way roughly in agreement with present theory for polymers under flow attachment.

Fluorescence labeling and imaging of micelles of several micrometres in length is now straightforward with hydrophobic fluorescent dyes. In addition, controlling the mean length of such soft and fluid assemblies is readily achieved by fragmentation in extrusion through nanoporous filters. By exploiting these methods, it has been shown that PEGylated filomicelles persist in the circulation considerably longer than any known spherical particles. Although filomicelles enter cells under static conditions, flow opposes entry into cells. As a result all processes have proven to be dependent on the length of filomicelles (requiring stable fluorescence labeling for such assessments).

The hydrophobic fluorescent dye used in these studies is widely used for long-term cell tracking in vivo (Oldernborg et al., Science 288: 2051-2054 (2000)). Present tests confirmed that when used to label filomicelles in whole blood in vitro for one week (at 37° C.) that (1) dye intensity is constant, (2) dye does not transfer to label blood cell membranes, and (3) filomicelles exhibit a constant length distribution. When visualized in blood samples taken from injected mice, filomicelles appear as freely diffusing, distinct and flexible cylinders, and their relative number, N/Nₒ, in each sample is therefore reliably determined by standard particle counting image analyses. Persistent circulation is seen for filomicelles that are inert or degradable, and the entire amount injected appears to be dispersed and sustained in the circulation of mice within 1-2 min.

A mathematical model based on a constant rate of filomicelle scission shows initial increases in N/No, before removal from the circulation. Such a mechanism is confirmed by the reduction of filomicelle length as the filaments circulate. Fluorescence allows for the measurement of filomicelle length in each sample (down to an optical resolution of ˜0.3 mm) and reveals a progressive decrease in length over one week, even when starting with inert filomicelles of moderate length. The initial shrinkage rate of ˜1 μm d⁻¹ is due to a combination of cell- and flow-induced fragmentation, and this rate appears to slow with time.

The following synthetic amphiphiles of many kilograms per mole in molecular weight are capable of self-assembling into filomicelles in aqueous solution. Preferred PEO-PEE block copolymers range in molecular weight from 1400 to 8700, with hydrophilic volume fraction, f_{EO}, ranging from 20% to 50%. For example, OCL1 (EO₄₆-CL₂₄; Mₙ 4.8 kg/mol; f_{EO} 0.42, core thickness 9.3 nm); OCL2 (EO₁₀₉-LA₅₆; Mₙ 10.0 kg/mol; f_{EO} 0.49, core thickness 11.4 nm); OCL3 (poly(ethylene oxide)-poly(ε-caprolactone) (PEO 5 k-PCL 6.5 k), and OB18 (EO₈₀-CL-_{125;} Mₙ 10.4 kg/mol; f_{EO} 0.29, core thickness 14.8 nm), wherein EO is ethylene oxide. For PEG-PCL, (EO₅₂-CL₄₄; molecular wt (kg/mol)=7; polydispersity=1.3 and PEG wt fraction=0.29); for PEG-PBD, (EO₂₆-CL₄₆; molecular wt (kg/mol)=3.6; polydispersity=1.09 and PEG wt fraction=0.33); for OS (poly(ethylene oxide)-b-polystyrene) (Mₙ=8000-9500, f_{EO}=0.46, polydispersity=1.07); for OE7′ ((EO₄₀-CL₃₇; molecular wt (kg/mol)=3.9; f_{EO} 0.39, core thickness 9 nm). Mₙ=number-average molecular weight, and PD=polydispersity index. Mean hydrophobic molecular weight Mₕ≈Mₙ−f_{EO}), and chain lengths of the hydrophobic blocks provide a simple scaling for membrane core thickness as d˜Mₕ.

While these compounds are exemplary of the synthetic amphiphiles suitable for use in the present invention, the list is not intended to be limiting, and many molecular variables can be altered with these illustrative polymers. Hence, a wide variety of material properties are available for the preparation of the filomicelle assemblies. One of ordinary skill in the art will readily recognize many other suitable block copolymers that can be used in the preparation of filomicelles based on the teachings of the present invention.

Degradable filomicelles of OCL3 exhibit a similar, but more sustained decrease in length, which is consistent with progressive shortening by hydrolysis. More rapidly degrading OCL1 filomicelles with a diameter similar to that of the inert filomicelles (e.g., comprising the OE7 copolymer) tend to disappear faster from the circulation (data not shown), which is also consistent with in situ hydrolysis. The shortest filomicelles (<4 μm) are seen to shorten somewhat slower than longer filomicelles, and 18 μm (Lₒ) filomicelles decrease most rapidly, fragmenting to 8 μm after just one hour in circulation. Subsequent circulation proves identical to the 8 μm filomicelles; as noted, this length approximates the diameter of rodent red blood cells, which circulate for many weeks. Because fragmentation does not double the particle numbers, the longer segments (of ˜10 μm) appear to be cleared from the circulation. Water-soluble nanotubes that are cleared in hours appear to be many micrometres in length, meaning that the difference with the present filomicelles might reflect rigidity more than length.

A simple binding isotherm fit of the circulation results for the filomicelles indicates a maximum half-life of about five days, and implies persistent circulation for soft cylinders with Lₒ>2.5 μm. The mononuclear phagocytic system (MPS) of the liver and the spleen constitutes the usual filtration and clearance pathway for circulating particulates and vesicles, as well as for the filamentous Ebola and H5N1 viruses. Fluorescence imaging of organ slices has shown that the liver and spleen also dominate the (slow) clearance of filomicelles. The degradable (hydrolyzable) polymer systems (OCL1 (EO₄₄-CL₂₄; f_{EO} 0.42, hydrated diameter 22 nm); and OCL3 (EO₁₁₀-CL₅₈; f_{EO} 0.43, hydrated diameter 60 nm)) show somewhat less mass in the spleen and a measurable accumulation in the kidney above tissue autofluorescence levels. The latter appears consistent with hydrolytic degradation leading to molecular-sized products that might permeate the fine mesh of the kidneys.

For drug delivery, the worm micelles of the present invention are shown to be able to incorporate a range of hydrophobic drugs into the cores of the worm-like micelles, and methods have been provided in, e.g., U.S. Pat. No. 6,835,394; U.S. Ser. No. 10/882,816 and Publ. US Pat. Appl. 2005/0180922, to chemically modify the ends of the PEO blocks to make the worm-like micelles specifically bind to suitable surfaces and cells. An enormously wide range of hydrophilic or hydrophobic materials can be associated with or encapsulated within a worm micelle, e.g., proteins and proteinaceous compositions and other antigens, therapeutics and other biomaterials that may enhance the immuno-response of the patient to a drug or vaccine, etc. The present invention, therefore, provides worm micelles which operate as adjuvants, or which encapsulate one or more “active agents,” which include, without limitation compositions, such as a drug, therapeutic compound, protein or protein fragment, gene or gene fragment, product of genetic engineering, or other antigenic composition suitable for achieving or enhancing the patient response to a selected therapeutic vaccine or immunotherapy, including specific anti-tumor therapies.

TAX is an anticancer drug that executes its chemotherapeutic effect by inhibiting the microtube movement to prevent the cell mitosis (Gligorovand et al., Oncologist 9:3-8 (2004)). Therefore, the drug, like many anticancer drugs, has to be delivered intracellularly into cytoplasm, and then remain there instead of being blocked or expelled from the cancer cells. This raised the concern as to whether the filomicelles can enter the tumor cells although they can escape from the blood vessels.

It is known that blood vessels in tumors are characteristically “leaky,” having pores (ranging from the tens to hundreds of nanometers) that allow nanoparticles to escape and accumulate in the solid tumors. Moreover, long filomicelles have been shown to persist in the circulation of mice for up to one week, whereas PEG vesicles injected as controls were cleared in two days. Interestingly, longer structures (˜18 μm) were very quickly fragmented by flow effects and phagocytic activity of the cells. This led to an optimal length that is approximately the same as the diameter of a biconcave red blood cell (8 μm), which are known to circulate for weeks. Conversely, shortened filomicelles (˜2 μm), fragmented more slowly and were much more gradually cleared from the blood. Thus, the filomicelle nanocarriers offer a new pathway for delivery of an active agent to cellular targets, and release of the encapsulated material.

Since filomicelles feature extended length (several microns) and often increased stiffness, it is definitely not realistic to think that they go through the popular endocytosis pathway, which would involve their encapsulation into endosomes which have only an approximately 500 nm diameter. Although it is possible that soft filomicelles may coil into a spherical shape to fit the endosome, that mechanism is clearly not energy-favored.

However, the studies performed in this invention confirmed that the stiff filomicelles, e.g., those comprising PEO-PS (OS) do, in fact, enter tumor cells, but before doing so, the assemblies undergo fragmentation and dissociation into significantly shorter filomicelles or even spheres with the suitable size. However, the resulting fragments or spheres did not behave in the same way as originally-formed spherical vesicles, and they appear to offer a different morphology and properties. As a result, filomicelles efficiently deliver drugs intracellularly, despite their special morphology. As OS filomicelles have a higher stiffness than OCL3 micelles (Vijayanand et al., supra, 2006), the dissociation of OCL3 filomicelles is also more efficient. Thus, the cellular entry of OCL3 filomicelles occurs within the scale of hours, if not faster.

In addition, TAX delivered by OCL3 polymeric micelles, whether worms or spheres, does alter the pharmacodynamic properties of TAX itself, as the tumor biodistribution and apoptosis index profiles consistently get higher with worm-like micelles, but lower with spherical micelles. Consequently, the dose-effect and pharmacodynamic profiles demonstrated the effective delivery of the solubilized drug. Moreover, when compared to Cremophor® EL, the currently commercial TAX formulation that has been reported to produce severe side effects including hypertension and nephrotoxicity. By comparison, the filomicelle system is entirely formulated using FDA-approved, fully biocompatible PEO-PCL copolymers when operating as a hydrophobic, anticancer drug carrier.

By “biocompatible” is meant a substance or composition that can be introduced into an animal, particularly into a human, without significant adverse effect. For example, when a material, substance or composition of matter is brought into a contact with a viable white blood cell, if the material, substance or composition of matter is toxic, reactive or biologically incompatible, the cells will perceive the material as foreign, harmful or immunogenic, causing activation of the immune response, and resulting in immediate, visible morphological changes in the cell. A “significant” adverse effect would be one that is considered sufficiently deleterious as to preclude introducing a substance into the patient.

As used herein, the term “induction of apoptosis” means a process by which a cell is affected in such a way that it begins the process of programmed cell death, which is characterized by the fragmentation of the cell into membrane-bound particles that are subsequently eliminated by the process of phagocytosis. Preferably apoptosis by the present invention is specific to the cancer cells being targeted (“bioselective”), without inappropriate apoptosis of the surrounding noncancerous tissue. “Inappropriate” or “unintended” apoptosis of normal (noncancerous) cells refers to apoptosis (i.e., programmed cell death) which occurs in cells of an animal at a rate different from the range of normal rates of apoptosis in cells of the same type in an animal of the same type, and which is increased or equal to the apoptosis seen in the cancerous cells. The terms “induced,” “enhanced,” “increased,” “inhibited,” “prevented” and the like are given their ordinary dictionary meanings with regard to disease therapies. For example, “enhanced” or “increased” apoptosis refers to an increase and/or induction of selective apoptosis of cancer cells. Conversely “inhibiting” cancer cell growth or “reversing” or “decreasing” tumor size means decreasing the relative tumor size or amount of cancer cells in at least one parameter, as compared to the size before or without treatment.

Degradable OCL3 micelles are about ten times stiffer (l,_{OCL3}≈5 mm) than inert micelles, but both circulate for a week or more, so flexibility would seem important, but weak, in its effects. To make truly solid cylindrical micelles, cross-linking was introduced into the core of the inert filomicelles, but when injected, these solid cylinders were found to be cleared within hours, which is similar to findings for water soluble, rigid carbon nanotubes of 30-38 nm in diameter. Circulation times thus seem set by the ability of a fluid cylinder micelle to relax and/or fragment, either in flow or because of interactions with cells.

Filomicelles as Nanocarriers. Carrier-mediated delivery of drugs into the cytosol is often limited by either release from the carrier or release from an internalizing endolysosome. But this problem is avoided by the use of the filomicelle nanocarriers. In the examples that follow, in vivo studies demonstrate growth arrest and shrinkage of rapidly growing tumors after intravenous administration of drug-carrying filomicelles comprising poly(ethylene glycol)-polyester.

Increasing the copolymer hydrophilic fraction, f_{EO}, by a mere 10% is sufficient to transform a membrane-forming block copolymer in water into one that forms a micelle over a range of PEG-polyester chains shortened by hydrolysis. Copolymers with small f_{EO} that self-assemble into continuous bilayer structures appear inert and devoid of any significant interactions with an adjacent lipid bilayer. However, copolymers with higher f_{EO} self-assemble into micellar morphologies, and, in time, spherical micelles interact strongly with the lipid bilayers. PEG arms extend toward the lipids as the polar lipid headgroups provide a favorable environment for transient PEG insertion. Note that although PEG is generally considered hydrophilic, it is known to exhibit surfactant-like activity in partitioning to an air:water interface at micromole per liter concentrations, and it also binds the headgroups of surfactants, thus stabilizing the micelles. The weak tethering that develops, is followed by copolymer transfer of the hydrophobic tails from the micelle into the lipid membrane, which increases until the whole micelle integrates and mixes into the lipid membrane. As expected with small detergents, curvature imparted by the micelle leads to pores in the membrane through which water—and soluble drug—will diffuse.

PEG appears to open these pores and facilitate permeation offer hypothetical mechanism for drug delivery using micelles of PEG-PCL. While tests have shown that while self-assembled membranes of copolymer and self-assembled membranes of lipid interact very little, very similar copolymers assembled into spherical micelles will fuse, mix, and porate a lipid membrane. Significantly, the functionality of these nano-structures can be enhanced if their in vivo degradation rates can be controlled by utilizing a physiologically benign stimuli, such as pH, light, redox, noise or heat. The sensitivity of the worm micelle structure to the environmental reduction potential is brought about by coupling of the hydrophobic and hydrophilic blocks. The synthesis and degradation of diblock copolymer worm micelles have been demonstrated using a di-block copolymer in which the hydrophilic block is polyethylene oxide and hydrophobic block is polycaprolactone.

Worm-like and spherical micelles were prepared herein from the same amphiphilic diblock copolymer, poly(ethylene oxide)-b-poly(ε-caprolactone) (PEO [5 kDa]-PCL [6.5 kDa]) in order to compare loading and delivery of hydrophobic drugs. Worm-like micelles of this degradable copolymer are nanometeric in cross-section and spontaneously assemble to stable lengths of microns. The highly flexible worm-like micelles can also be sonicated to generate kinetically stable spherical micelles composed of the same copolymer. The fission process exploits the finding that the PCL cores are fluid, rather than glassy or crystalline, and core-loading of the hydrophobic anticancer drug delivery, paclitaxel (TAX) shows that the worm-like micelles load and solubilize twice as much drug as spherical micelles. In cytotoxicity tests that compare to the clinically prevalent solubilizer, Cremophor® EL, both micellar carriers are far less toxic, and both types of TAX-loaded micelles also show 5-fold greater anticancer activity on A549 human lung cancer cells. PEO-PCL based worm-like filomicelles appear to be promising pharmaceutical nanocarriers with improved solubilization efficiency and comparable stability to spherical micelles, as well as better safety and efficacy in vitro compared to the prevalent Cremophor® EL TAX formulation.

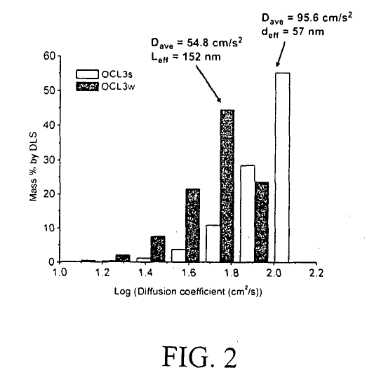

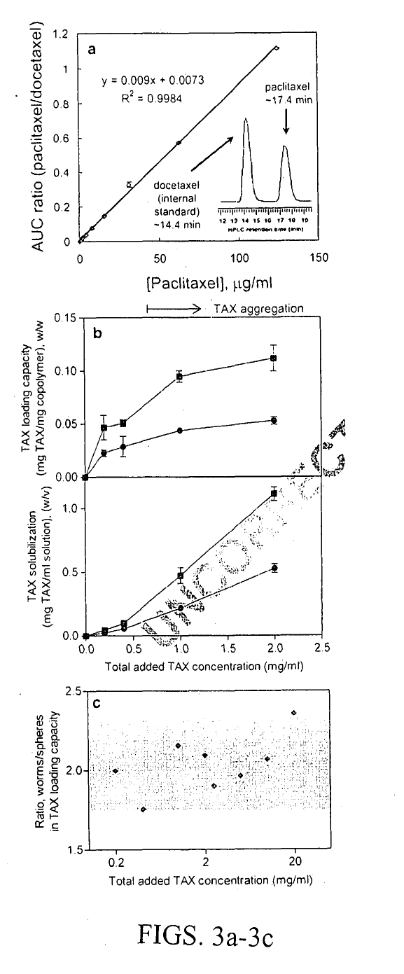

The needed copolymers typically comprise ˜0.4-0.5 weight fraction of hydrophilic PEO polymer and yield flexible, but highly stable, filamentous micelles that surprisingly increase the circulation time in the blood stream relative to spheres. As shown herein, filomicelles generally have a fluid core (Example 1), which favors integration of drugs into their hydrophobic cores, leading to an examination of the drug loading capacity and various other performance aspects of PEO-PCL worm-like filomicelles for comparison to spheres generated from the same filomicelles by sonication. Loading advantages are clear (FIG. 3B, 3C, Tables 2, 3 and 4), and some insight is gained from simple calculations of volume to surface area for spheres and cylinders.

For spheres of radius r, volume/surface area=(4/3 πr³)/(4 πr²)=r/3, whereas for cylinders of length L, volume/surface area=(πr²L)/(2 πrL)=r/2. If micellar area is thus held constant for a given mass of copolymer in solution, then the filomicelles are expected to carry more hydrophobic drug than spheres by a ratio of (r/2−r/3)/(r/3)=50%. The bigger difference of ˜100% found in the present data certainly highlights the advantageous loading of drugs into filomicelles. Furthermore, the maximum concentration of solubilized TAX reached 3 mg/ml, placing filomicelles just below Cremophor EL (marketed as formulations containing 6 mg/ml TAX), but among the top micelle-based TAX delivery systems that usually enhance TAX solubility to 1-2 mg/ml (see, Soga et al., supra, 2005; Cheon Lee et al., supra, 2003; Moand et al., supra, 2005; Liu et al., supra, 2005; Xie et al., supra, 2005, Kim et al., supra, 1999).

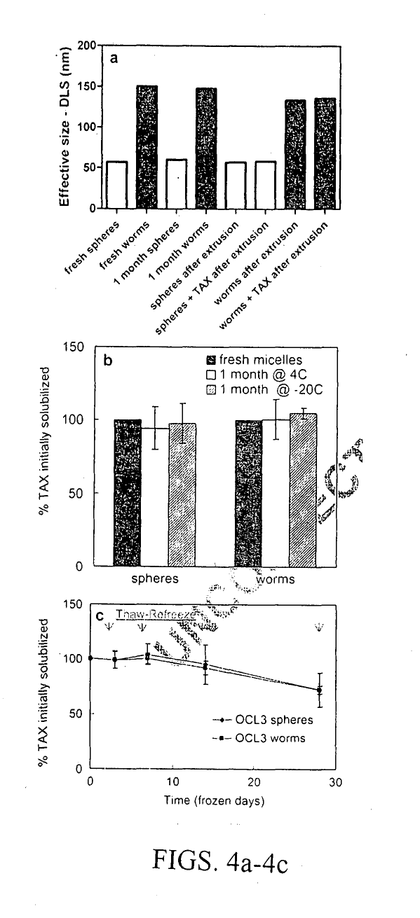

Although filomicelles appear novel and were perhaps overlooked in past formulations that relied on sonication, PEO-PCL block copolymers have been widely explored for drug delivery applications in the past. It consists of FDA-approved PEO plus the approved and degradable polyester PCL, wherein hydrolytic degradation of PCL predominates at the distal hydroxyl-end, such that the hydrophobic mass of PCL gradually decreases, increasing the weight fraction of PEO, eventually reducing the worm-like micelles to the size of spherical micelles (Geng et al., supra, 2005). This process is greatly accelerated by lowering the pH, which also accelerates release of TAX. On the other hand, degradation of PCL is significantly limited at low temperatures of 4° and −20° C., and the present results demonstrate stable storage of OCL3 filomicelle morphologies at these low temperatures for a month (FIG. 4A-4C) or more (2 months, 3 months, 4 months 6 months, 9 months, 1 year, >1 year) without major complications from crystallization. While multiple freeze/thaw cycles leads to loss of TAX activity for both worm-like and spherical micelles, as would any other harsh conditions in extrusion, sonication, and lyophilization (not shown), minimal exposure (only 1-2 cycles of freeze/thaw cycles) has no obvious effect. Care should nonetheless be taken when preparing or storing TAX-loaded worm micelles.

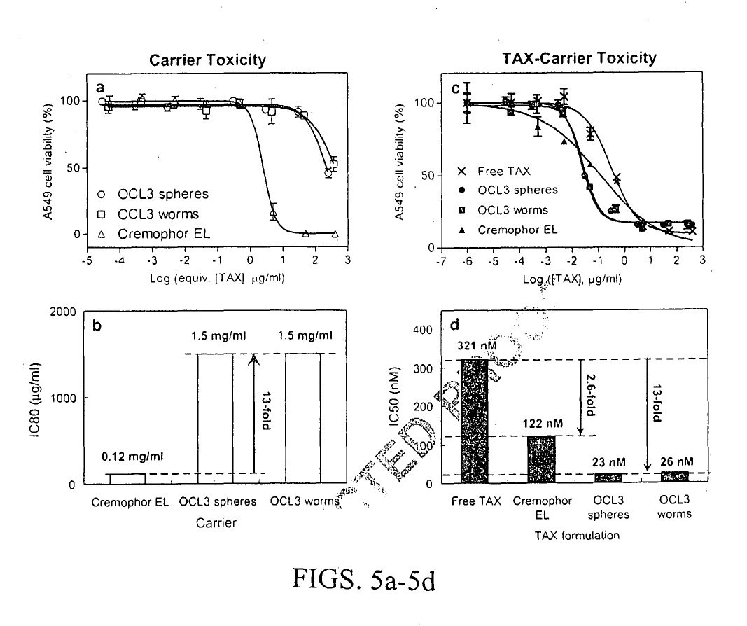

Given the persistent circulation of filomicelles and minimal accumulation in rat lung (Cheon Lee et al., supra, 2003), specific targeting of these novel morphologies to lung tumors was evaluated (Example 2), thus providing a clear indication of directed drug delivery possibilities using filomicelle nanocarriers. Human lung cancer also continues to account for a significant amount of all cancer deaths. Accordingly, with these factors in mind, and with knowledge of the intrinsic toxicity of Cremophor EL (FIG. 5A, 5B), the in vitro delivery by TAX filled filomicelles to A549 lung carcinoma cells, the present data show that the spherical micelles and the filomicelles are both 13-fold less toxic than Cremophor EL, yet when loaded with TAX, they are about 5-fold more effective in delivering a cytotoxic dose (FIG. 5C, 5D). Furthermore, since delivery of Cremophor EL is not pH-sensitive, such an in vivo formulation will tend to be less selective for tumors and further increase the risk of the excipient toxicity to normal cells.

Dosage and Delivery. In one aspect of the invention, a compound (active agent) is assessed for therapeutic activity following delivery by the filomicelle methods disclosed herein, by examining the effect of the compound as described and exemplified herein or by any recognized assay method. Controls may include the assay mixture without the test compound or delivery system and the assay mixture having the test compound or delivery system. The mixture is incubated for a selected length of time and temperature under conditions suitable for delivery and therapeutic effect of the active agent following encapsulation and delivery as described herein, whereupon the reaction is stopped and the effectiveness of the delivery method and of the released active agent, or an absence of activity is assessed, also as described herein.

Compounds that induce apoptosis or shrink the size of a tumor following delivery by the filomicelle system, either by enhancing or inhibiting the activity, are easily identified in the assay by assessing therapeutic effects by the methods exemplified in the presence or absence of the test compound are readily assessed. A reduction in tumor size or reduced growth of the tumor, following administration of the filomicelle carrier and encapsulated drug, as compared with the absence of the test compound in the assay, is an indication that the filomicelle delivery system is effective at delivering a known anticancer compound. Similarly, an increased, or significantly increased level, or higher amounts of apoptosis of the cancer cells in the presence of the test compound following administration of the filomicelle carrier and encapsulated drug, as compared with the absence of the test compound in the assay demonstrates that the filomicelle delivery system is effective at delivering a known anticancer compound intracellularly to the animal model or patient in vivo.

The method of the invention is not limited by the type of active agent or drug or other compound used in the assay. The test compound is thus a synthetic or naturally-occurring molecule, which may comprise a peptide or peptide-like molecule, or it is any other molecule, either small or large, which is suitable for testing in the assay. In another embodiment, the test compound is an antibody or antisense molecule, RNAi and the like directed against the cancer or tumor.

Compounds that inhibit cancer activity or growth or increase or enhance apoptosis of the cancer cells in vitro are further tested for similar therapeutic activity in vivo in humans, using the same biocompatible filomicelle delivery methods and systems described herein. Essentially, the compound is administered to the human as disclosed by any known route, but exemplified herein using I.V., and the effect of the released active agent is assessed by clinical and symptomatic evaluation. Such assessment is well known to the practitioner in the field of developmental biology or those studying the effect of cancer drugs. Compounds may also be assessed in an in vivo animal model, as herein described.

Precise formulations and dosages will depend on the nature of the test compound and may be determined using standard techniques, by a pharmacologist of ordinary skill in the art. The composition according to the invention is intended especially for the preventive or curative treatment of disorders, such as hyperproliferative disorders and cancers, including those induced by carcinogens, viruses and/or dysregulation of oncogene expression, specifically for treatment of neoplastic tumors. The treatment of cancer (before or after the appearance of significant symptoms) is particularly preferred, particularly for treatment of a cancerous tumor.

The phrase “therapeutically effective amount” is used herein to mean an amount sufficient to reduce by at least about 10%, or 15%, or 20%, or 25%, or by at least 50%, or by at least 90%, and most preferably complete remission of a hyperproliferative disease or cancer of the host or patient. Alternatively, a “therapeutically effective amount” is sufficient to cause an improvement in a clinically significant condition in the host. In the context of the present invention, a therapeutically effective amount of a drug at the minimal therapeutic dose, is that minimal and least toxic dose or amount which is effective to treat a proliferative disease or tumor or other cancerous condition, in a patient or host, thereby effecting a reduction in size or virulence or the elimination of such disease or cancer. Preferably, administration or expression of an “effective” amount of the active agent as delivered by the filomicelle methods and system herein, resolves the underlying infection or cancer or cancerous tumor.

A pharmaceutical composition according to the invention may be manufactured in a conventional manner. In particular, a therapeutically effective amount of a therapeutic or prophylactic agent is combined with a vehicle such as a diluent. A composition according to the invention may be administered to a patient (human or animal) by aerosol or via any conventional route in use in the field of the art, especially via the oral, subcutaneous, intramuscular, intravenous, intraperitoneal, intrapulmonary, intratumoral, intratracheal route or a combination of routes. I.V. dosage is preferred. The administration may take place in a single dose or a dose repeated one or more times after a certain time interval.

The appropriate administration route and dosage vary in accordance with various parameters, for example with the individual being treated or the disorder to be treated, or alternatively with the drugs, therapeutic active agents or gene(s) of interest to be transferred. The particular formulation employed will be selected according to conventional knowledge depending on the properties of the tumor, or hyperproliferative target tissue and the desired site of action to ensure optimal activity of the active ingredients, i.e., the extent to which the encapsulated active agent reaches its target tissue following delivery by the filomicelle methods and system herein, or by assessing a biological fluid from which the filomicelle carrier or the encapsulated drug or the like has access to its site of action. In addition, these viruses may be delivered using any vehicles useful for administration of the above identified drug, compound etc., which would be known to those skilled in the art. It can be packaged into capsules, tablets, etc. using formulations known to those skilled in the art of pharmaceutical formulation.

Dosages for a given host can be determined using conventional considerations, e.g., by customary comparison of the differential activities of the subject preparations and a known appropriate, conventional pharmacological protocol. Generally, a pharmaceutical composition according to the invention comprises a dose of the drug, protein, etc. according to the invention of between 10⁴ and 10¹⁴, advantageously 10⁵ and 10¹³, and preferably 10⁶ and 10¹¹.

The formulations of the pharmaceutical compositions described herein may be prepared by any method known or hereafter developed in the art of pharmacology. In general, such preparatory methods include the step of bringing the active ingredient into association with the filomicelle carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, and kidney dialytic infusion techniques.

Formulations of a pharmaceutical composition suitable for parenteral administration comprise the encapsulated drug, protein, active agent or the like which prior to encapsulation has been combined with a pharmaceutically acceptable carrier, such as sterile water or sterile isotonic saline. Then the filomicelle delivery system containing the encapsulated drug, protein, active agent and the like is further formulated for delivery to the patient by combining it with a pharmaceutically acceptable carrier, such as sterile water or sterile isotonic saline. Thus, in the present invention, it is the filomicelle delivery system that forms the pharmaceutical composition suitable for parenteral administration. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to encapsulation and parenteral administration of the reconstituted composition.

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The pharmaceutical compositions may be prepared, packaged, or sold in the form of a sterile, injectable, aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

The formulations described herein are also useful for pulmonary delivery and the treatment of such cancers of the respiratory system or lung, and are also useful for intranasal delivery of a pharmaceutical composition of the invention. Such formulation suitable for intranasal administration is a coarse powder comprising the active ingredient and having an average particle from about 0.2 to 500 micrometers, administered by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

As used herein, the term “pharmaceutically-acceptable carrier” expressly means the filomicelle delivery methods and systems disclosed herein, and used to deliver a drug, protein, active agent, or other chemical composition and the like to a mammal.

The pharmaceutical compositions useful for practicing the invention may be administered to deliver a dose of between 1 ng/kg/day and 100 mg/kg/day. In one embodiment, the invention envisions administration of a dose which results in a therapeutically effective concentration of the drug, protein, active agent, etc., between 1 μM and 10 μM in a diseased or cancer-affected tissue, or tumor of a mammal when analyzed in vivo.

A pharmaceutical composition, especially one used for prophylactic purposes, can comprise, in addition, a pharmaceutically acceptable adjuvant, carrier, fillers or the like. Suitable pharmaceutically acceptable carriers are well known in the art. Examples of typical carriers include saline, buffered saline and other salts, liposomes, and surfactants. The adenovirus may also be lyophilized and administered in the form of a powder. Taking appropriate precautions not to denature the protein, the preparations can be sterilized and if desired mixed with auxiliary agents, e.g., lubricants, preservatives, stabilizers, wetting agents, emulsifiers, salts for influencing osmotic pressure, buffers, and the like that do not deleteriously react with the active virus. They also can be combined where desired with other biologically active agents, e.g., antisense DNA or mRNA.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject, or a convenient fraction of such a dosage, such as, for example, one-half or one-third of such a dosage, as would be known in the art. For cancer therapies, such dosage of the drug, protein or active agent and the like being delivered by the filomicelle delivery methods and systems would be readily determined by an oncologist or others familiar with such drug, protein or active agent and the like.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

The compositions and methods described herein can be useful for preventing or treating cancers including leukemias, lymphomas, meningiomas, mixed tumors of salivary glands, adenomas, carcinomas, adenocarcinomas, sarcomas, dysgerminomas, retinoblastomas, Wilms' tumors, neuroblastomas, melanomas, and mesotheliomas; as represented by a number of types of cancers, including but not limited to breast cancer, sarcomas and other neoplasms, bladder cancer, colon cancer, lung cancer, pancreatic cancer, gastric cancer, cervical cancer, ovarian cancer, brain cancers, various leukemias and lymphomas. One would expect that any other human tumor cell, regardless of expression of functional p53, would be subject to treatment or prevention by the methods of the present invention, although the particular emphasis is on mammary cells and mammary tumors. The invention also encompasses a method of treatment, according to which a therapeutically effective amount of the drug, protein, active agent, etc., or a vector comprising same according to the invention is administered to a patient requiring such treatment. The invention should not be construed as being limited solely to these examples, as other cancer-associated diseases which are at present unknown, once known, may also be treatable using the methods of the invention.

Also useful in conjunction with the methods provided in the present invention would be chemotherapy, phototherapy, anti-angiogenic or irradiation therapies, separately or combined, which maybe used before or during the enhanced treatments of the present invention, but will be most effectively used after the cells have been sensitized by the present methods. As used herein, the phrase “chemotherapeutic agent” means any chemical agent or drug used in chemotherapy treatment, which selectively affects tumor cells, including but not limited to, such agents as adriamycin, actinomycin D, camptothecin, colchicine, taxol, cisplatinum, vincristine, vinblastine, and methotrexate. Other such agents are well known in the art.

As described above, the agents encompassed by this invention are not limited to working by any one mechanism, and may for example be effective by direct poisoning, apoptosis or other mechanisms of cell death or killing, tumor inactivation, or other mechanisms known or unknown. The means for contacting tumor cells with these agents and the effect of the selected chemotherapeutic agent on a subject are well known and readily available to those of skill in the art. The novelty lies in delivery by the filomicelle methods and systems disclosed herein.

Also, radiation or surgery may be used in conjunction with the treatments disclosed herein. The term “irradiation” or “irradiating” is intended in its broadest sense to include any treatment of a tumor cell or subject by photons, electrons, neutrons or other ionizing radiations. These radiations include, but are not limited to, X-rays, gamma-radiation, or heavy ion particles, such as alpha or beta particles. Moreover, the irradiation may be radioactive, as is commonly used in cancer treatment and can include interstitial irradiation. The means for irradiating tumor cells and a subject are well known and readily available to those of skill in the art.

The present invention is further described by example. These examples are provided for purposes of illustration only, and are not intended to be limiting unless otherwise specified. The various scenarios are relevant for many practical situations, and are intended to be merely exemplary to those skilled in the art. These examples are not to be construed as limiting the scope of the appended claims, rather such claims should be construed to encompass any and all variations that become evident as a result of the teachings provided herein.

### Examples

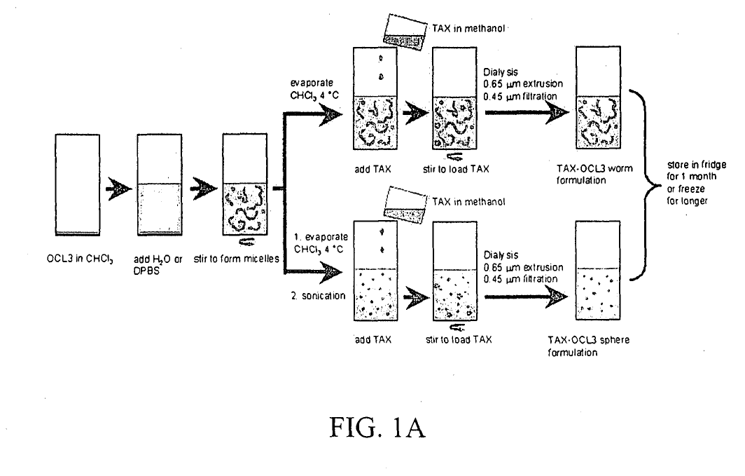

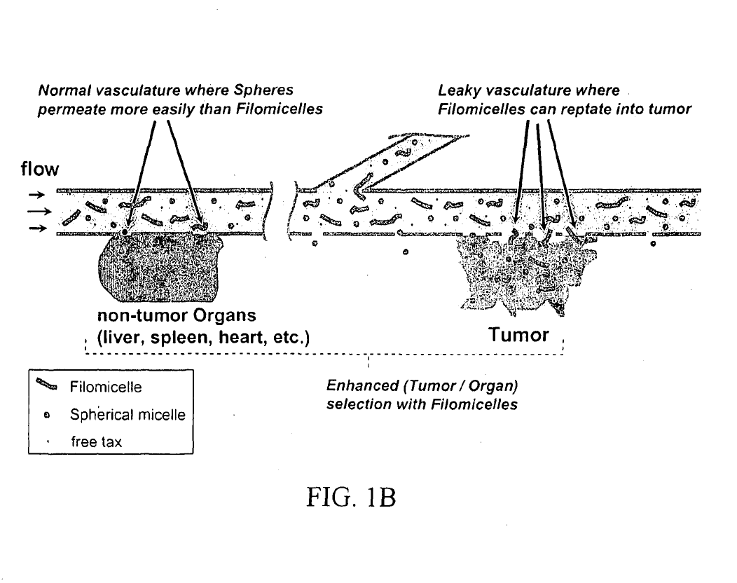

To examine the ability to deliver TAX in vivo, specifically into the tumors of tumor-bearing mice, an FDA-approved diblock polymer poly(ethylene oxide)-b-poly(ε-caprolactone) (PEO 5 kDa-PCL 6.5 kDa, denoted “OCL3”)-based worm-like filomicelles and spherical micelles were prepared. Moreover, due to the enhanced permeation and retention (EPR) effect of tumor sites where leaky vascular structure is prevalent (see, Liu et al., supra, 2006), there was further evaluation to determine whether filomicelles were more inclined to “reptate” into the tumors, rather than into other non-tumor organs that featured tight vasculatures (FIG. 1B). Unlike spherical micelles, worm-like filomicelles have to reptate through the pores on the blood vessels in limited directions. Therefore, once they have entered the cell, it would be much more difficult for worm-like filomicelles and their encapsulated contents, as opposed to spherical micelle delivery vehicles, to escape from the blood vessels in normal organs.

Materials. Diblock polymer poly(ethylene oxide)-b-poly(ε-caprolactone) (Mₙ=5000-6500, weight fraction of PEO f_{EO}=0.43, polydispersity=1.3, denoted OCL3) and poly(ethylene oxide)-b-polystyrene (Mₙ,=8000-9500, f_{EO}=0.46, polydispersity=1.07, denoted OS) were from Polymersource, Inc. (Dorval, Canada). Paclitaxel (TAX), docetaxel, fluorescent PKH26 dye, and Dulbecco's phosphate-buffered saline, pH 7.4 (DPBS) were from Sigma (St. Louis, Mo.). Ncr nude mice (female, ˜5 weeks, 20 g) were purchased from Taconic Farms, Inc. (Germantown, N.Y.). Cell death detection ELISA^{plus} kit was from Roche (Indianapolis, Ind.). Human lung carcinoma cells A549 were from ATCC (Manassas, Va.). F12 Ham media was from Mediatech, Inc. (Herndon, Va.). All organic solvents were analytical grade from Fisher Scientific.

Preparation of OCL3 Polymeric Micelles by Co-solvent Evaporation Method. The preparation of OCL3 polymeric micelles is shown in FIG. 1A. Briefly, 50 μl of 50 mg/ml OCL3 stock solution in chloroform was mixed with 5 ml of water and the mixture was stirred vigorously at room temperature for 1 h. Chloroform was completely removed by evaporation at 4° C. overnight to obtain the final solution containing OCL3 worm-like micelles. Solutions at other concentrations were made by varying the volume of OCL3 stock solution mixed with water. OCL3 spherical micelles were obtained by sonicating the worm-like micelles using Fisher 60 Sonic Dismembrator equipped with Fisher Ultrasonic Converter (Fisher Scientific) for 25 pulses at 1 sec/pulse. All solutions containing OCL3 polymeric micelles were stored at 4° C. to minimize degradation.

Fluorescence Microscopy and Micelle Morphology Studies. PKH26, which is a rhodamine-based hydrophobic fluorescent dye, was added to OCL3 micelle solutions at about 1 ml/1 mg polymer and vortexed for 10 sec. The dye rapidly distributed into the hydrophobic core of the micelles so the morphology of micelles was visualized.

Olympus IX71 inverted fluorescence microscope equipped with a 60× objective lens and a Cascade CCD camera was used to observe the micelles. About 25 images were taken for each sample tested and the contour length of the worm-like micelles was measured using Image-Pro Plus (MediaCybernetics, Silver Spring, Md.).

Dynamic Light Scattering. The average hydrodynamicmicelle sizes and size distribution were analyzed by dynamic light scattering (DLS) using Protein Solutions™ Temperature Controlled MicroSampler and Protein Solutions Dynapro™ Titan (Wyatt Technologies, Santa Barbara, Calif.) at 25° C. The laser wavelength was 782.4 nm and the scattering angle was 90°. Each sample was measured in triplicate.

In a size and diffusion analysis of the OCL3 micelles, the average diffusion coefficient distribution, and the calculated effective hydrodynamic size (diameter or length) of worm-like (before sonication) and spherical (after sonication) micelles were measured by DLS as shown in FIG. 2. The calculation of effective length of worm-like micelles is based on the Stokes-Einstein equation:

$$\begin{matrix}D = \frac{kT}{( 2 πη {L}_{eff} )} for worms & Equation 1 \\ D = \frac{kT}{( 3 πη {d}_{eff} )} for spheres & Equation 2\end{matrix}$$

where D is the diffusion coefficient, k is the Boltzmann constant (1.38×10⁻²³ joules per kelvin (J/K), T is the temperature (25° C.), η is the viscosity of the solution (1.02 mPa·s from DLS), and d_{eff} is the hydrodynamic radius multiplied by 2. See, Li et al., Phys. Rev., E Stat. Nonlin. Soft Matter Phys. 69:______ (2004).

Crystallinity Analysis. Polycaprolactone is a highly crystalline polymer in bulk. In an attempt to look for crystallization at nano-scale, an alternate protocol was developed that could exploit the melting and annealing of the PCL core. Thus, a 10 mg/ml stock solution of OCL3 was prepared in chloroform and 15-20 ul of this stock was added to 1 ml water in a glass vial. The vial was closed, briefly vortexed, and allowed to stand at room temperature for 2 h. Next, the vial was incubated at 60° C. with the cap open, for 2 h to evaporate the chloroform in the solution. The vial was then allowed to incubate at 30° C. for 4-6 h. Glycerol was added to the filomicelles to make a 50% glycerol solution, which was incubated at −20° C. for up to 24 h.

The rigidity of OCL3 worms was determined by fluorescence image analysis as described in “Fluorescence Microscopy and Micelle Morphology Studies” above.

The fluidity of the worm micelle core was estimated by measuring the fluorescence recovery after photobleaching (FRAP) of the PKH26 dye. Briefly, after photobleaching (FRAP) curve on OCL3 worms: an aperture on the light path is used to selectively overexpose a small section of the worm. The fluorescence recovery in the bleached region was monitored by comparing the fluorescent intensity to that of the Intensity in an unbleached section of the worm.

The intensity in the bleached section was compared to that of a similar length of the worm in the unbleached section to normalize for bleaching during imaging. The FRAP data were fitted to an exponential recovery curve:

Recovery %=A(1−e^{-t/t}) Equation 3

where A is the maximum recovery percentage and t is the time for recovery) to obtain an average recovery time constant τ ˜28 sec. The 1-D diffusivity of the PKH26 dye was calculated from D=L_{b}²/2τ where L_{b} is the length of the bleached region, and τ is the recovery time constant. D ˜0.9 μm²/s. A percentage of rigid OCL3 worm-like micelles with possibly crystallized cores was measured over 12 hours of incubation at −20° C. either in a 50% glycerol solution or pure water, after heating to 60° C. and cooling to 30°. From the resulting data a differential scanning calorimetry (DSC) thermogram was calculated (not shown) ranging from 25° to 80° C. of OCL3 polymer alone, and as a TAX-OCL3 mixture.

Paclitaxel (TAX) Encapsulation in OCL3 Micelles. TAX of 50 mg/ml in methanol was added into the micelle solutions to obtain desired spiked TAX/polymer ratios. The mixture was stirred at 25° C. for 20 min and transferred to dialysis cassettes (MWCO 10,000, Pierce, Rockford, Ill.). Dialysis was performed against DPBS (pH 7.4) for 2 h to remove methanol and small residues of dissolved TAX, and the obtained TAX-loaded micelles were separated from insoluble-free TAX aggregates by extrusion through a 10 ml thermobarrel extruder (Northern Lipids, Vancouver, Canada) fitted with 0.65 μm filtering membranes (Millipore, Bedford, Mass.). This was further purified by filtration through 0.45 um Fischerbrand MCE filter (Fisher Scientific). As previously described, the preparation of TAX-encapsulated OCL3 micelles is illustrated in FIG. 1A, but as an alternative method, TAX was mixed with OCL3 polymer in chloroform solution before micelle formation. The TAX-loaded micelles were then obtained as described in “Preparation of OCL3 Polymeric Micelles by Co-solvent/Evaporation Method” above, followed by dialysis and extrusion.

HPLC Assay Development and Validation. A WatersHPLC system (Waters, Milford, Mass.) equipped with a 1525 Binary HPLC pump®, a symmetry\reverse-phase C₁₈ 5.0 mm column (4.6×150 mm), and a 2487 Dual λ absorbance UV detector was used for TAX quantification. A series of 1:2 TAX dilutions in acetonitrile ranging from 0 to 0.25 mg/ml were pre-mixed with equal volume of 0.25 mg/ml docetaxel in acetonitrile as internal standard. The solutions were filtered through 0.45 um filter followed by injection into HPLC system. A mobile phase of 58% H₂O, 42% acetonitrile at a flow rate of 1 ml/min was applied. TAX was detected and quantified at UV 220 nm. The standard curve by plotting the ratio of AUC of TAX and docetaxel was established, and the linear range, intra-day and inter-day coefficient of variance (CV), lower limit of detection (LLOD), lower limit of quantification (LLOQ), assay accuracy and recovery (by testing with 3, 10, and 40 mg/ml TAX solution using the standard curve) were calculated. To use the validated HPLC assay to determine the TAX loading capacity and efficiency in OCL3 micelles, TAX-loaded micelles were pre-mixed with equal volume of 0.25 mg/ml docetaxel in acetonitrile, and acetonitrile in equal volume to the mixture was further added, followed by the HPLC analysis using the standard curve described above. TAX loading capacity and efficiency were calculated based on the following expressions:

$$\begin{matrix}TAX loading capacity = \frac{mass \text{:} of \text{:} TAX \text{:} encapsulated \text{:} in \text{:} filomicelles}{mass : of \text{:} OCL 3 \text{:} polymeric \text{:} filomicelles} & Equation 4 \\ TAX loading capacity = \frac{massofTaxencapulatedinmicelles}{massofinitiallyaddedTAX} & Equation 5\end{matrix}$$

Thermal Analysis. Thermal tests of OCL3 micelles were performed by differential scanning calorimetry (DSC) using a Differential Scanning Calorimeter 2920 (TA instruments, New Castle, Del.).

TAX-loaded OCL3 worm micelles were lyophilized before the analysis. DSC thermograms of OCL3-TAX mixture (either in bulk before TAX loading or in lyophilized form after TAX loading) and OCL3 alone were then obtained by heating in sealed standard aluminum pans (TA instruments) from 25° to 100° C. at a rate of 10° C./min followed by air cooling and reheating to 100° C. at the same rate.

Micelle Stability and Paclitaxel Release Studies. Both worm-like and spherical micelles (10 mg/ml), either drug-loaded or free, were stored at 4° C. for 1 month or subjected to a one-time freeze/thaw cycle. Then, the particle size was measured by DLS and the morphology was tested by fluorescent microscopy. TAX-loaded micelles were also examined for drug leakage potentially caused by storage or freeze/thaw cycles by centrifugation at 3,000 rpm for 5 min to precipitate the TAX that may have diffused out. The supernatant was then mixed with acetonitrile and internal standard docetaxel for HPLC analysis.

Further, a dialysis method was employed to evaluate the in vitro release of TAX from OCL3 micelles. TAX-loaded worm-like and spherical micelles at a TAX concentration of 40 μg/ml were added to the dialysis cassettes and dialyzed at 37° C. against DPBS of pH 6.8 and pH 7.4. At certain time points, the release medium was sampled and fresh DPBS was added to maintain the volume. The sampled medium was lyophilized and redissolved in chloroform. The insoluble buffer salt was removed by filtration. Chloroform was evaporated then and the remaining sample was re-dissolved in acetonitrile and subjected to aforementioned HPLC analysis.

Cytotoxicity Assay. TAX-loaded and drug-free micelles at serial dilutions were prepared as above. For comparison, 12 mg/ml TAX in ethanol was mixed with an equal volume of Cremophor® EL followed by sonication for 30 min. The resulting Cremophor EL TAX was diluted with DPBS to obtain desired TAX concentrations. Human lung-derived carcinoma cells A549 were grown in F12 Ham media supplemented with 10% fetal bovine serum and 100 U/ml penicillin and 100 μg/ml streptomycin at 37° C., 5% CO₂ to 60-70% confluence. A549 cells (50,000 cell/ml) were seeded in 96-well plates at 5,000 cells per well and cultured for 24 h to allow attachment. The medium was then exchanged and 100 μl of different tested formulations (free worm-like and spherical OCL3 micelles, Cremophor EL, free drug, TAX-loaded worm-like and spherical micelles, and Cremophor EL TAX) was added. As control, 100 ml of DPBS was added to cells not exposed to those formulations. After 37° C., 5% CO₂ incubation for 72 h, the media were discarded, and 100 μl/well F12 Ham medium and 11 μl/well of 5 mg/ml MTT solution in DPBS was added. The plates were incubated at 37° C. for 3 h and the media were removed again. The intracellular metabolized product MTT formazan was retrieved by addition of 100 μl/well DMSO and incubation at room temperature for 5 min. The plates were read at 550 nm, and the cell viability was calculated as (reading of wells with cells exposed to tested formulations minus the reading of the blank wells) divided by (reading of wells with cells exposed to DPBS minus the reading of the blank wells).

Data Analysis. All data that require non-linear regression analysis were processed using GraphPad Prism (Version 4.03, GraphPad Software, San Diego, Calif.). The contour length distribution of OCL3 worm-like micelles was fit by Gaussian distribution, TAX and the carrier cytotoxicity assay on A549 cells was fit by sigmoid dose-response curve equation.

### Example 1

### Advantages of PEO-PCL Filomicelles in TAX Delivery

OCL3 Filomicelles are Fluid and can Fission to Spheres. A simple physicochemical measure of aggregate stability for strongly segregating amphiphiles is the critical micelle concentration (CMC), which was expected to be exponential in the length of the hydrophobic block (Vijayanand et al., supra, 2006). Based on a CMC of 1.2 mg/ml for a sphere-forming OCL copolymer, EO₄₄-CL₂₁ (Luo et al., Bioconjug. Chem. 13:1259-1265 (2002)), an immeasurably small CMC was estimated for the present OCL3 copolymer EO₁₁₀-CL₅₈ of less than 1 femtogram (fg)/mL (i.e., CMC_{OCL3}˜[1.2 μg/ml^{58/21}). For later comparison, Cremophor EL reportedly has a CMCCremEL ˜90 mg/ml. For OCL3, micellar assemblies are clearly the predominant form in any aqueous solution. Moreover, since molecular exchange rates between micelles scale inversely with CMC, these low-CMC micelles can be considered to be kinetically trapped or frozen, without implying glassiness or crystallinity.

The weight fraction of OCL3 ˜0.43 for the hydrophilic PEO block, drives assembly of most of the copolymer into worm-like and flexible filomicelles, as observed by fluorescence microscopy after adding hydrophobic fluorescent dyes (not shown).

The average contour length of spontaneously assembled OCL3 filomicelles was calculated from measurement of at least 50 filomicelles. Most of the filomicelles are 6-7 μm, with some filomicelles as long as 14 μm. Extrusion of worm-like filomicelles at high pressures and flow rates through nanoporous filters has been used to controllably reduce their length (Geng et al., supra, 2007 in press), but in order to convert to spherical micelles simply and quantitatively, the filomicelles were exposed to robust sonication for several minutes. Diffusion coefficients (Dₐᵥₑ) were then measured by dynamic light scattering (DLS), and after sonication the effective hydrodynamic diameter was ˜57 nm (FIG. 2). This is similar to previous ˜60 nm estimates for the OCL3 filomicelle diameter of core plus corona as based on cryo-TEM (Id.). Prior to sonication, the measured Dₐᵥₑ proves significantly smaller, and yields only a crude approximation for a larger effective size, but more important is the minimal overlap of the two distributions for Dₐᵥₑ. The 22% overlap suggests that a small fraction at most of the pre-sonicated sample consists of spherical micelles. Worm-like filomicelles, therefore, predominate in the freshly prepared OCL3 samples.

In bulk, polycaprolactone (PCL), specifically b-poly(ε-caprolactone) is a crystalline polymer at room temperature with melting in the range of 40°-60° C. (Skoglundand et al., J. Appl. Polym. Sci. 61:2455-2465 (1996)), but past studies of PEO-based diblock copolymers in bulk suggest that PEO crystallization dominates in the same temperature range and frustrates crystallization of the connected block (Id.). For example, the diblock PEO-polyethylene (PE) in bulk is 70% PEO and only 10% crystalline (PE). On the other hand, dilution of filomicelles into water will generally hydrate and dissolve any crystallinity in the PEO corona. Previously OCL filomicelles were reported to appear as flexible as worm-like micelles made from non-crystalline and non-glassy diblock copolymers (e.g., PEO-polybutadiene), which implies a fluid core of PCL (Geng, et al. supra, 2005; Balsamo et al., Macromolecules 36:4507-4514 (2003)). However, because the presently stored filomicelles used in these experiments involved freezing for an extended time, which are conditions that favor crystallization, the fluid core was reconfirmed.

A very small fraction of the OCL3 filomicelles are inflexible coils as identified by end-to-end fluctuation <5% of the average end-to-end distances relative to the more fluid-like and flexible filomicelles. Freezing in glycerol gave up to 10% of the bent, but crystalline-behaving worm-like micelles. The core fluidity of the dominant population of flexible filomicelles was subsequently estimated by fluorescence recovery after photobleaching (FRAP) studies of immobilized filomicelles. The average recovery time for four different worms was ˜30 s, which is similar to that observed in polybutadiene (PBD) cores of worm-like micelles with a similar molecular weight (Geng et al., Angew. Chem., Int. Ed. Engl. 45:7578-7581 (2006)). The fast recovery rate in FRAP and the minor percentage of rigid worm micelles indicate a highly fluid PCL core, which tend to favor loading and retention of hydrophobic drugs such as paclitaxel (TAX). Fluidity also provides a basis for filomicelle flexibility, allowing these long micelles to reptate into diseased tissues, including tumors, despite their micron-scale length.

Before examining TAX loading of filomicelles and spheres in dilute solution, the bulk melting of OCL3, with or without TAX, was examined using differential scanning calorimetry (DSC). A melting onset temperature that was observed near 51° C. is consistent with PEO and/or PCL crystallization, and the finding that TAX exerted no significant effect on melting temperature indicates a relative absence of disruptive interactions between the drug and the copolymer. Assuming the melting is attributable predominantly to PEO crystallization, as cited above, PEO crystallinity was estimated to be 81% from the measured endotherm peak area (106.3 J/g without TAX), the heat of fusion for pure PEO (˜300 J/g) (Hillmyer et al., Macromolecules 29:6994-7002 (1996)), and the f_{EO} of OCL3. With the presence of TAX, there was no apparent decrease of crystalline PEO (79%) in bulk OCL3 (102.4 J/g with TAX), suggesting that TAX interaction with the copolymer is negligible.

Paclitaxel Integration into OCL3 Filaments and Shortened Filomicelles. TAX was then loaded into dilute micelles, and HPLC analysis was used to characterize the loading properties. An internal standard, docetaxel, was added in fixed amount to minimize variability (FIG. 3A). The lower limit of detection (LLOD) and lower limit of quantification (LLOQ) were as low as 0.5 and 1 μg/ml, and both the intra-day and inter-day coefficients of variance (CV) were less than 15%. An accuracy of 100.2±7.8% (n=6) and a recovery of 385 101.7±6.1% (n=6), were also obtained.

Loading of TAX (defined as mg TAX loaded per milligram micelle FIG. 3A, plotting the HPLC-UV-determined area-under-curve (AUC) ratio between TAX and docetaxel vs TAX concentration before or after micelle formation), showed no significant difference in capacity or efficiency. FIG. 3B shows TAX loading capacity (defined as mg TAX loaded per milliliter aqueous solution) and solubilization with OCL3 micelles, which varied when the total added TAX:OCL3 micelle (w/w) was fixed at 1:5 when added TAX concentration≦2 mg/ml, and OCL3 micelle concentration was fixed at 10% (w/v) when added TAX concentration≧2 mg/ml. The inset (FIG. 3C) shows the ratio between TAX loading capacity with OCL3 worms and spheres at different conditions. Total added TAX:OCL3 micelle (w/w) was fixed at 1:5 while OCL3 concentration varied was fixed at 1:5 while OCL3 concentration varied when added TAX concentration≦2 mg/ml, and OCL3 micelle concentration was fixed at 10% (w/v) when added TAX concentration≧2 mg/ml. As a result, increasing TAX from 0.2 to 2 mg/ml (OCL3 1-10 mg/ml) increased both the loading capacity of TAX and the final solubilized TAX. Compared to spheres, OCL3 filomicelles showed about 2-fold greater loading capacity and at all concentrations (FIG. 3C).

Up to 10% w/v polymer concentration, with TAX varied from 2.5 to 5 mg/ml, the loading capacity for TAX increased, although the w/w loading was higher for 5% polymer. Further increases of added TAX up to 20 mg/ml led to a decrease in TAX solubilization regardless of morphology; this was probably due to the well-known aggregation of TAX at extremely high concentrations. The highest TAX solubilization obtained in the studies of filomicelles to date was 3 mg/ml, which is about 10,000-fold higher than natural TAX solubility in aqueous buffer [0.3 μg/ml, (Soga et al., supra, 2005)]. Drug loading efficiency defined as (loaded drug/added drug) is tabulated in Tables 1, 2, and 3 and consistently appears to be 2-fold higher with filomicelles than with spherical micelles.

TABLE 1
Loading Efficiency of TAX into OCL3 Spherical And Worm-like
Micelles When Initial Added TAX: OCL3 Micelle (w/w) = 15.
Total TAX Concentration (mg/ml)

| | 0.2 | 0.4 | 1 | 2 | 20 |
|---|---|---|---|---|---|
| Spheres | 0.11 | 0.14 | 0.22 | 0.26 | 0.03 |
| Worms | 0.23 | 0.25 | 0.47 | 0.56 | 0.06 |

TABLE 2
Loading Efficiency of TAX into OCL3 Spherical and Worm-like Micelles
at 10% (w/v) Fixed OCL3 Micelle Concentration.
Total TAX Concentration (mg/ml)

| | 2.5 | 5 | 10 | 20 |
|---|---|---|---|---|
| Spheres | 0.34 | 0.30 | 0.09 | 0.03 |
| Worms | 0.64 | 0.59 | 0.18 | 0.06 |

TABLE 3
Loading Efficiency of TAX in Worm-Like OCL3 Micelles at 5 and
10% (w/v).
Total TAX Concentration (mg/ml)

| | 2.5 | 5 | 10 |
|---|---|---|---|
| Spheres | 0.44 | 0.49 | 0.17 |
| Worms | 0.64 | 0.59 | 0.18 |

In addition to the encapsulation capacity studies, an identical DSC thermogram for lyophilized OCL3-TAX mixture after encapsulation (not shown) with that of OCL3-TAX mixture in bulk again verified the unchanged melting temperature and the fusion heat of OCL3 copolymer. This further demonstrated the absence of interactions of TAX with its excipient during the encapsulation process.

Stability and In Vitro Release. Possible effects of storage, drug loading and extrusion on morphological changes of filomicelles were examined by DLS and fluorescence imaging. FIG. 4A shows that (1) both shapes of micelle are morphologically stable in 4° C. storage for up to 1 month; (2) TAX integration does not affect micelle size, which is probably because the small loaded mass of TAX cannot swell the relatively large cores within the filomicelles; and (3) spherical micelles made by sonicating worm-like filomicelles show no further size change after extrusion, whereas filomicelles become smaller. The latter result was confirmed by contour length measurement under fluorescence microscopy, which showed that extrusion shifts and narrows the length distribution from 6.6-7.3 um (95%) to 5.6-6.1 um.

Stability of TAX loading within OCL3 micelles was evaluated after 1 month of storage. FIG. 4B shows that when TAX-loaded micelles of either morphology were either maintained in fluid form at 4° C., or else frozen (and perhaps crystalline) at −20° C., no significant leakage or precipitation of TAX was detected. As emphasized above and further below, the filomicelle carriers are clearly stable under harsh treatments. For application, freezing has the advantage in slowing hydrolytic degradation of PCL.

In addition, filomicelles were subjected to freeze/thaw cycles (−20° C.) as another potentially disruptive operation relevant to storage. After a single cycle, there was no significant change in the length distribution shown in TAX retention (FIG. 4C). However, the plot in FIG. 4C shows that multiple, repeated freeze/thaw cycles do eventually lead to drug leakage from both morphologies. TAX-loaded OCL3 micelles were frozen at day 0, then thawed at day 3 for the test, and then re-frozen, which was repeated at day 7, 14, and 28. By the fourth cycle, the TAX retained in the micelle cores decreased to about 70% of the amount initially loaded. The reasons are not yet as clear as the practical implications.

Release in vitro at 37° C. was also studied at pH 7.4, per normal tissue pH, and also at pH 6.8 to mimic the slightly acidic cancerous tissue environment (Gao et al., supra, 2005; Webb et al., Clin. Exp. Metastasis. 17:397-407 (1999)). As the PCL in OCL3 is known to undergo accelerated hydrolysis at acidic pH (Geng et al., supra, 2005), TAX release rates proved to be 40% faster at lower pH, but similar for both morphologies. This indicates that pH rather than shape is the more critical parameter to control drug release.

Enhanced Cytotoxicity of TAX Released from OCL3 Micelles. Human lung carcinoma-derived A549 cells were used in cytotoxicity assays of both micelles as empty carriers and also as TAX-loaded carriers. The in-clinic, commercial TAX formulation with Cremophor EL was also included as a benchmark. Excipient toxicity is critically important to assessing the specific anticancer effect of TAX, and so for ease of comparison we therefore calculate the equivalent TAX concentration; for example, 0.8 mg TAX corresponds to about 1 g Cremophor EL (see “Materials” above). Based on such analyses, Cremophor EL shows a significant cytotoxic effect at dosages as low as only 2-3 μg/ml equivalents of TAX (FIG. 5A). In contrast, the OCL3 polymeric micelles showed no obvious toxicity at dosages up to almost 100 μg/ml TAX equivalents. Identifying the dose of excipients at which 80% of cells are still alive (“inhibition constant” IC80) as a parameterization of toxicity and then converting to cytotoxic carrier doses yields IC80_{CremEL}=120 μg/ml for Cremophor EL, which appears only slightly higher than CMC_{CremEL}≈90 μg/ml and implies the aggregate form of Cremophor EL is toxic (FIG. 5B). For both filomicelles and spheres, IC80_{OCL3-micelle}=1,500 μg/ml, which is about 13-orders of magnitude higher than CMC_{OCL3} and suggests mechanisms of cell death such as micellar poration previously discussed for PEO-PCL polymersomes (Geng et al., supra, 2005). Regardless, the 13-fold difference indicates, of course, that OCL3 polymeric micelles are much safer excipients.

TAX formulations with the various carriers consistently improve cytotoxicity relative to delivery of free drug. While FIG. 5C shows that TAX-loaded Cremophor EL begins killing cells in the nanogram/milliliter range of TAX and kills nearly all of the cells at [TAX]=10-100 μg/ml (FIG. 5A), the Cremophor EL excipient rather than TAX is clearly responsible for a significant fraction of the cytotoxicity. On the other hand, TAX-loaded OCL3 micelles exhibited similar cytotoxicity in the 10 ng/ml range, killing more than 35% A549 cancer cells. Importantly, the anticancer effects of TAX-loaded OCL3 micelles throughout the tested concentrations were all attributed to the drug activity, instead of the toxicity from the carriers. The fitted sigmoid dose-response curve showed that the IC50-cytotoxicity of TAX-loaded OCL3 micelles (at ˜25 nM) was 13-fold more potent than free TAX (321 nM for A549 cells) and also 5-fold better than Cremophor EL TAX (FIG. 5D). Both worm-like filomicelles and spherical micelles showed the same enhanced cytotoxicity. Since OCL3 filomicelles have a higher drug loading capacity as compared to spherical micelles, and otherwise display similar stability and efficacy as compared to spherical micelles, the filomicelles offer an attractive method for parenteral delivery. However, to optimize the effectiveness and efficiency of the filomicelle delivery system, the maximum tolerated dose had to be determined.

### Example 2

### Maximum Tolerated Dose (MTD) Determination

OCL3 polymeric micelles and filomicelles, and TAX encapsulation procedures, were conducted as described by Geng et al., supra, 2005B; and Cai et al., supra, 2007. A series of concentrations of TAX-loaded OCL3 micelles in DPBS solution were prepared as 0.5, 1, and 2 mg/ml (corresponding to 5, 10, and 20 mg/kg for 20 g mice with 0.2 ml injection per animal) for spherical micelles and 0.5, 1, 2 and 3 mg/ml (corresponding to 5, 10, 20, and 30 mg/kg for 20 g mice with 0.2 ml injection per animal) for filomicelles. The same concentrations of empty OCL3 micelles were also prepared. Nrc nude mice were weighed followed by a single tail vein injection of either empty OCL3 micelles and TAX-loaded micelles in different concentrations at 0.2 ml per mouse. After 24 hours, the mice were weighed again. The “maximum tolerated dose” (MTD) was determined to be, and defined as, the dose that caused the mouse body weight loss<10%.

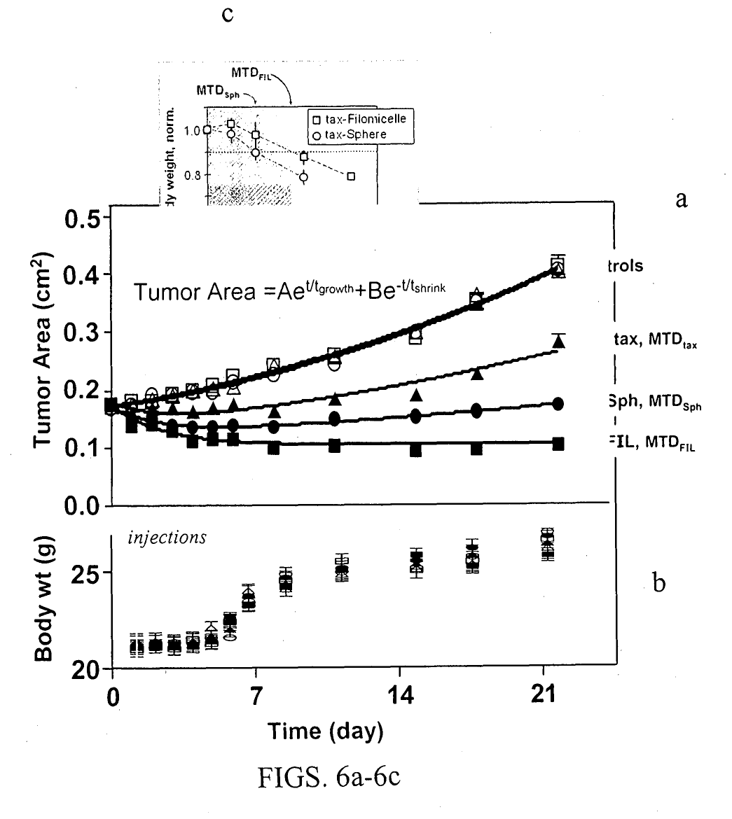

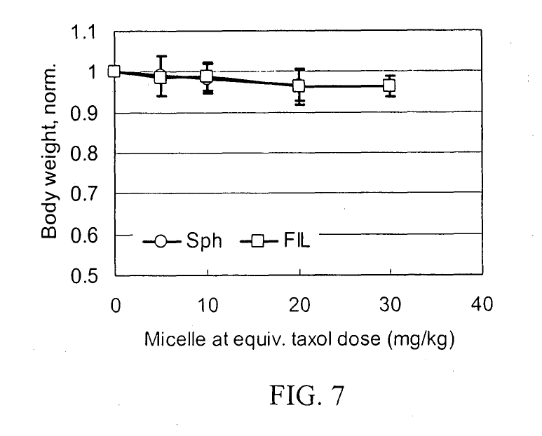

As shown in FIG. 6A, both the TAX-loaded OCL3 spherical micelles and the worm-like micelles caused the body loss with increased doses (FIG. 6B), but no body weight loss was observed with the polymeric carriers alone throughout the tested range (FIG. 7). Importantly, mice injected with TAX-loaded OCL3 worm-like micelles showed an enhanced tolerance (MTD ˜18 mg/kg) against the anticancer drug, as compared with the ones injected with TAX-loaded spheres (MTD ˜10 mg/kg). The 80% enhancement of MTD of TAX delivered with worm-like filomicelles permits a higher dosage to be administered into the mice, thus leading to a better therapeutic effect. Also as seen in FIG. 7, OCL3 polymeric micelles are highly safe carriers, showing no toxicity, even at an injection dose up to 30 mg/kg TAX equivalent (corresponding to about 300 mg/kg OCL3 polymer). Thus, by determining the maximum tolerated dose (MTD) for each administered anticancer drug, such as paclitaxel (TAX), when delivered by OCL3 micelles, the maximal anticancer effect could be achieved at the tumor site, without requiring high dosages.

Tumor shrinkage studies. Human lung carcinoma cell line, A549, was injected subcutaneously at MTD per mouse. After about 3 weeks, the tumor-bearing mice were injected with TAX-loaded OCL3 micelles at MTD of the corresponding morphology, as well as with free TAX at MTD (1 mg/kg), and with DPBS saline alone. The same injections were repeated after 3, 7, 10, 14, 17 and 21 days. Tumor area (A) was monitored 24 hrs after each injection by measuring two orthogonal dimensions as A=[(L₁×L₂)/2] for each treatment group. The body weight of the mice was also monitored throughout the experiment.

The tumor shrinkage effect of different carriers was also examined in a multiple-injection experiment. To guarantee that the animals were safe during the experiment, 16 mg/kg TAX was selected as the injected MTD for filomicelles, and 8 mg/kg TAX was administered using the spheres. TAX in DPBS solution was also used for comparison, with the MTD of 1 mg/kg injected. FIG. 6C illustrates that during the 21-day experimental period, the tumor-bearing mice were all healthy with steady body weight gains, indicating that all the TAX formulations were reasonably safe to the animals. It should be noted here that the weight gaining due to tumor growth (if possible) for all groups was minimal, wherein the largest tumor area gained after 3 weeks was 0.4 cm² (FIG. 6A), corresponding to an approximate volume of 0.2 cm³ (assuming the tumor was spherical in shape). This would have contributed to only 0.2 g of the total weight (assuming the density of tumor is 1 g/ml).

On the other hand, as shown in FIG. 6A, the tumor growth was significantly inhibited with TAX administration compared to the control group, wherein only DPBS saline was injected. Free TAX injection at MTD showed a modest inhibition of tumor growth, whereas TAX encapsulated with OCL3 micelles shrank the tumor to a much higher degree. Notably, the mice administered OCL3 worm-like filomicelles carrying TAX at MTD almost showed no sign of tumor size increase throughout 22 days, which was substantially better than any other treatment groups.

As a better comparison, TAX delivered by filomicelles was also injected at a same dose as MTD of spheres. This resulted in a similar tumor shrinkage outcome (data not shown), indicating that it is the drug dosage, rather than the excipient form, that is crucial for determining the degree of tumor shrinkage that will result. Therefore, a significant increase of the MTD, which as shown above can be achieved using filomicelles, would be of benefit to anticancer therapies.

To evaluate the tumor inhibition effect of the different TAX delivery methods in a more quantitative way, the TAX-inhibited tumor growth vs. time plot was fit by applying an exponential model (Simeoni et al., Cancer Res. 64:1094-101 (2004)), incorporating both a tumor growth phase and a tumor shrinkage phase with the TAX addition. The modeling was performed by fit the parameters in the equation (Table 4):

Tumor Area=Σ Ae^{t/growth}+Be^{-t/shrink} Equation 7

where t is the time (day), A and t_{growth} are constants of the tumor growth phase, and B and tₛₕᵣᵢₙₖ are constants of the TAX-inhibition phase.

TABLE 4
List of the constants in modeling of the TAX-inhibited lung carcinoma
A549 cell growth in mice.

| | A (cm²) | t_{growth} (week) | B (cm²) | tₛₕᵣᵢₙₖ (week) | R² |
|---|---|---|---|---|---|
| Controls | 0.168 | 3.5 | 0.00 | ∞ | 0.990 |
| tax | 0.014 | 1.2 | 0.178 | 8.8 | 0.977 |
| tax-Sph | 0.118 | 3.5 | 0.050 | 0.36 | 0.879 |
| tax-FIL | 0.104 | 84 | 0.075 | 0.36 | 0.928 |

Table 4 shows that when TAX was absent (Control), the tₛₕᵣᵢₙₖ was infinitive (∞), indicating there was no tumor shrinkage effect. However, when TAX was delivered by growth polymeric micelles, the t_{growth} increased significantly from 3.5 weeks to more than 2 months (using spheres), and for nearly 2 years (using filomicelles), indicating that the tumor growth dramatically slowed, to the point of almost completely ceased when the TAX-filomicelle formulation was administered. On the other hand, the tₛₕᵣᵢₙₖ decreased from infinitive (∞) to only 2-3 days, showing the obvious effect of worm-like micelle-delivered TAX for killing and shrinking tumor cells. Although it seems that the tumor growth was enhanced with free TAX administration (the t_{growth} was actually reduced to about 9 days), but most probably the free TAX could shrink tumor by killing tumor cells (tₛₕᵣᵢₙₖ dramatically reduced from infinitive (∞) to 8 weeks) thus still control the tumor growth in a modest way as shown in FIG. 2.

Adjourned

However, when all of the TAX formulations were compared, it was apparent that filomicelle-delivered TAX has shown the best effect in both retarding tumor growth and killing tumor cells. Taken together, this data demonstrates that worm-like filomicelles could be used as a more effective and powerful carrier for TAX intravenous delivery with enhanced tolerated dose to establish better tumor inhibition effect as compared to spherical micelles.

Cell apoptosis of tumor and non-tumor organs. Cell apoptosis is an important parameter to evaluate the death of the cells within a specific tissue or organ. Consequently, finding a high percentage of cell apoptosis in the tumor confirms the anti-tumor effect of the applied chemotherapy, while at the same time a relatively low percentage of apoptosis in other non-tumor organs of the subject animal ensures the administration of the drug will result in only mild side effects. Therefore, the cell apoptosis index was measured by the following procedure.

A DNA-based photometric enzyme immunoassay system (Cell Death Detection ELISA) was employed in accordance with the manufacturer's instructions to evaluate the cell apoptosis within organs. The mice, following I.V. administration of TAX by the methods described above, were sacrificed after 22 days and their organs, including liver, kidney, spleen, heart, lung and tumor were removed. Those organs were weighed and homogenized by Dounce Homogenizer (Belco Glass, Vineland, N.J.). The supernatant after centrifugation was then used to determine the specific enrichment of nucleosomes that had been released into the cytoplasm due to the death of the cells caused by the treatments. The cell apoptosis index was calculated by dividing the Enrichment factor of TAX treatment animal groups by the Enrichment factor of untreated animal groups (Enrichment factor of TAX treatment animal groups/Enrichment factor of untreated animal groups). The cell apoptosis index for the untreated groups was designated 1.

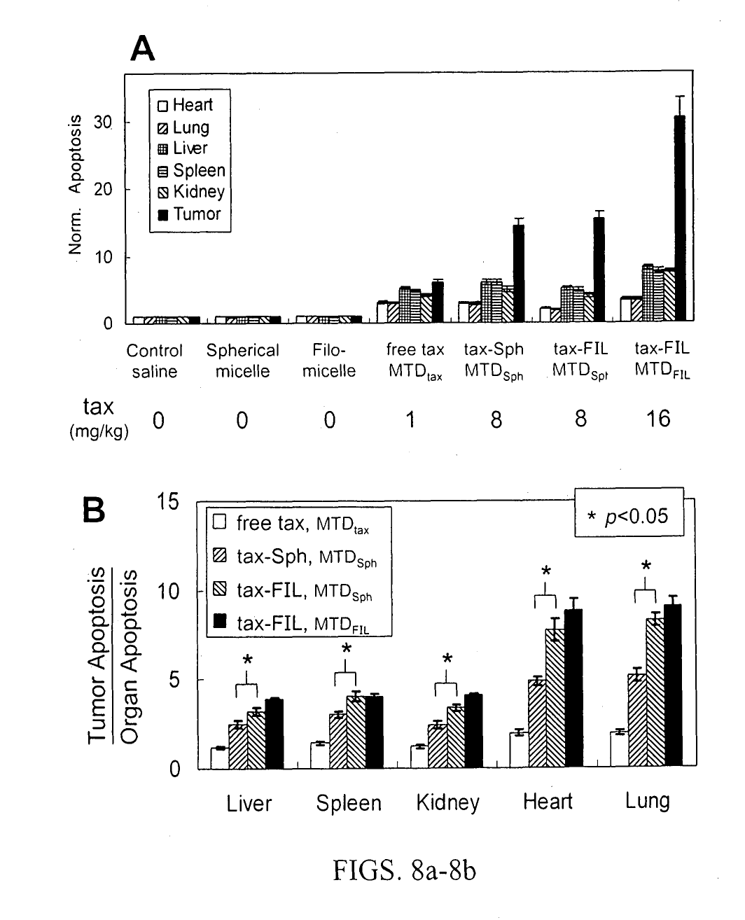

As clearly shown in FIG. 8A, within the six tested organs (liver, kidney, spleen, heart, lung and tumor), mice injected with DPBS or polymeric micelles alone did not present any detectable apoptotic cells. In contrast, TAX treated mice showed a significantly increased cell apoptosis index for all the organs, demonstrating the cytotoxic effect of the anti-neoplastic drug. Free TAX injection into the animals did not generate much difference of the cell apoptosis between tumor and other non-tumor organs, indicating that the administration of TAX-alone did not have a good tumor-specific effect. By comparison, TAX carried to the tumors by OCL3 filomicelles achieved a significant tumor to non-tumor organ cell apoptosis ratio, demonstrating a tumor-specific cell apoptotic result when the drug was delivered by the worm-like filomicelles.

Further comparison between TAX carried by spherical and filomicelles confirmed the better tumor selectivity of filomicelles in TAX delivery. At the same administered TAX dose of 8 mg/kg, although there was no apparent difference between the tumor cell apoptosis using these two type of carriers, the apoptosis index in other, non-tumor organs was 15-30% lower when delivered by filomicelles, as compared with delivery by spheres. Even when the TAX dose was doubled to 16 mg/kg, delivery by filomicelles only caused a modest increase (30-40%) in the apoptosis index in non-tumor organs, but it resulted in a proportional (2-fold) enhanced apoptosis in the tumor cells. No experiments were performed using spheres to deliver such a high TAX dose because that would have exceeded the MTD of spherical micelles—which was only 10 mg/kg, as shown above.

TABLE 5
Cell apoptosis index ratio between tumor and non-tumor organs in mice, 22
days after multiple I.V. injections of 8 mg/kg TAX encapsulated in spheres
(OCL3s) or by TAX encapsulated in worm-like filomicelles (OCL3w).

| TAX, | | | % Change between |
|---|---|---|---|
| 8 mg/kg | OCL3s | OCL3w | spheres and worms |
| Liver | 2.47 ± 0.20 | 3.19 ± 0.20 | +29% |
| Kidney | 3.07 ± 0.17 | 4.03 ± 0.26 | +31% |
| Spleen | 2.47 ± 0.21 | 3.40 ± 0.15 | +38% |
| Heart | 4.85 ± 0.26 | 7.74 ± 0.65 | +60% |
| Lung | 5.15 ± 0.34 | 8.31 ± 0.34 | +61% |

Accordingly, taking the ratio of cell apoptosis in tumor vs. in non-tumor organs, it was clearly seen that worm-like filomicelles enhanced the tumor-selective cytotoxicity of TAX by 30-60% at the same TAX dose of 8 mg/kg, as compared to spheres (FIG. 8B and Table 5). This may occur because the filomicelles, having an increased effective size may reptate into the leaky vascular structure of tumor to deliver TAX due to the “EPR (enhanced permeation and retention)” effect Liu et al., supra, 2006), whereas such worm-like vehicles may not easily permeate through the compact tissue of the blood vessel walls of other organs, and as a result less TAX is delivered to the non-tumor sites (see schematic in FIG. 1B).

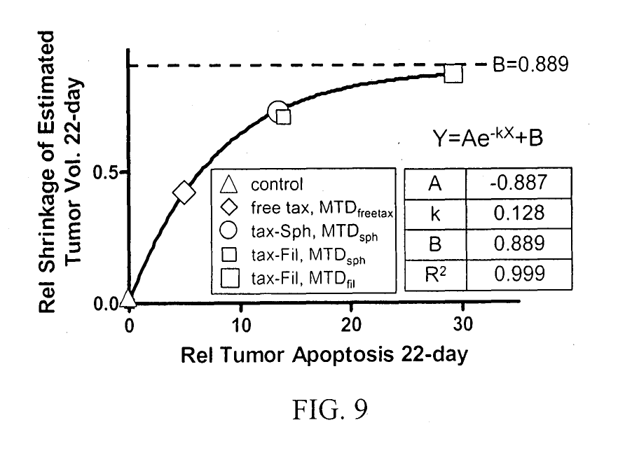

By plotting the relative shrinkage of the estimated tumor volume (calculated from the tumor area by assuming the implanted tumor was in a spherical shape) versus the relative tumor apoptosis at day 22 following the onset of TAX treatment, the relationship between apoptosis and tumor volume was fit by an exponential equation (FIG. 9). Relative shrinkage of estimate tumor volume was calculated as: (tumor size in control group−tumor size in treatment group)/(tumor size in control group). Tumor size (cm³) was estimated from the tumor area (cm²). It was shown that the relative shrinkage of the tumors (87%) when the mice are treated with TAX-encapsulated filomicelles had already closely approached the maximum shrinkage Y=Aₑ^{-kX}+B, wherein apoptosis inducement is 89%. Cell apoptosis inducement is the recognized pathway of killing tumor cells by paclitaxel (Millenbaugh et al., Pharm. Res. 15:122-7 (1998)), but evidence had indicated that tumors cannot be completely shrunk by cell apoptosis alone. This could be attributed to the development of drug-resistant tumor cells, and actually implies the limitation of chemotherapy—even delivery of elevated dose of paclitaxel by other methods (e.g., targeted delivery) may not completely remove the tumor and additional surgery is recommended for better prognosis.

Biodistribution studies. The distribution of TAX in mice was determined by HPLC method. Briefly, at 24 hours after a single tail vein injection of TAX-loaded OCL3 micelles and free TAX, the tumor-bearing mice were sacrificed and their liver, kidney, spleen, heart, lung and tumor were removed. Those organs were weighed, and homogenized in 1-2 ml 1:1 acetonitrile (ACN):H₂O with addition of 50 μl of 10 μg/ml docetaxel as the internal standard. The homogenate was then mixed with 5 ml ethyl acetate for 24 hrs. After centrifugation at 6000 rpm, 4° C. for 10 min., the supernatant was transferred to a new vial, dried under N₂ stream, and reconstituted with 200 μl ACN. The solution was filtered through 0.45 μm poly(tetrafluoroethylene) (PTFE) filter and then subjected to HPLC with the methods described above.

Studies of cancer cell uptake of worm-like filomicelles. To better understand the entry pattern of worm-like filomicelles into the tumor cells, the glassy worm-like micelles made of poly(ethylene oxide)-polystyrene (PEO-PS, or OS (Vijayanand et al., supra, 2006)) were incubated with A549 cells at 25 μl of 100 μg/ml OS filomicelles in DPBS (labeled with PKH26 red fluorescent dye, Ex: 551 nm; Em: 567 nm) for 50,000 cells in 0.5 ml F12 Ham media. The incubation was conducted at 37° C. At time points of 30 min, 1 hr, 2 hrs, and 4 hrs, both the filomicelle-containing media and the A549 cells were visualized under an Olympus IX71 inverted fluorescence microscope. The length of the OS filomicelles and the intracellular fluorescence intensity observed were analyzed by ImageJ software (see http://rsb.info.nih.gov/ij/).

a) TAX Delivered by Worm-Like Filomicelles Accumulated Higher in Tumors.

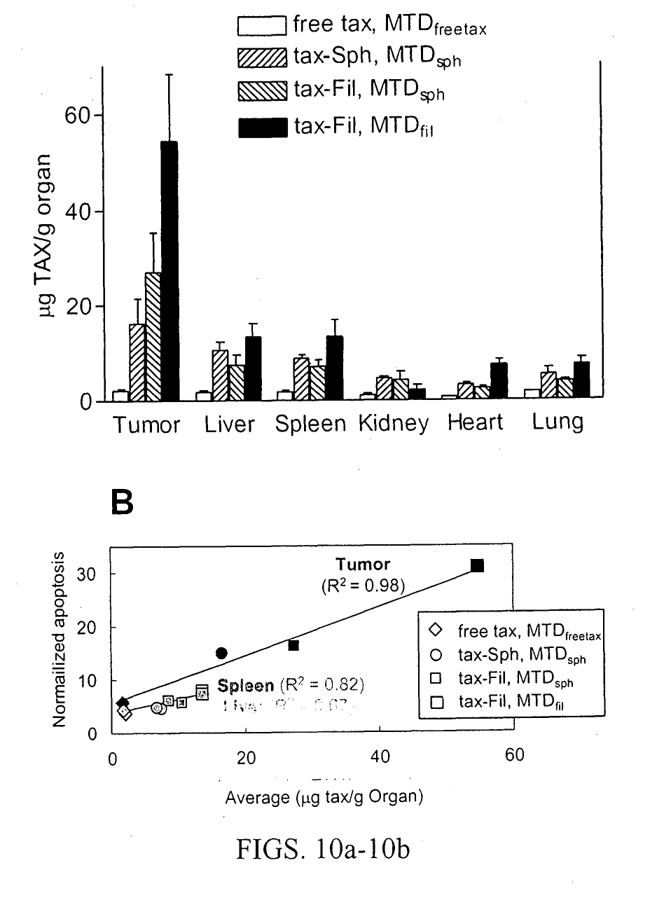

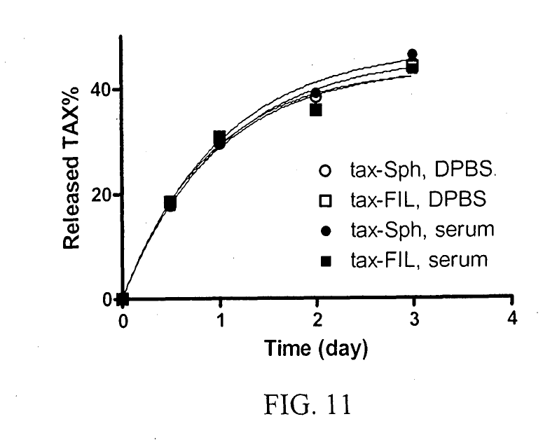

To better understand the distribution of TAX in vivo, A549 tumor cell-bearing mice injected with a single I.V. dose of TAX in different carriers were sacrificed after 24 hrs of the injection. The biodistribution of TAX in tumor and other major organs was determined by HPLC methods. As is shown in FIG. 10A, TAX delivered by polymeric micelles showed better tumor-selective accumulation of TAX than when TAX was delivered alone directly into the animal. Moreover, worm-like filomicelles further facilitated the tumor distribution of TAX as compared to delivery by spheres at the same dose of 8 mg/kg TAX, although not by significant amounts. Thus, the overall biodistribution profile is consistent with the higher tumor-specific apoptosis associated with filomicelle delivery as demonstrated above, and indicates that the accumulated dose-effect relationship of TAX was not virtually affected by using various carriers. Such pharmacodynamic properties were further demonstrated by plotting apoptosis value vs. TAX biodistribution in tumor, liver and spleen (FIG. 10B). It should also be noted that the release of TAX from OCL3 polymeric micelles was stable and independent of whether the TAX was in DPBS buffer (pH 7.4) or whether it was 1:1 DPBS:fetal bovine serum (FBS) as determined by a simple HPLC method of Millenbaugh et al., supra, 1998. The addition of serum had no effect on the TAX release rate (FIG. 11).

b) Even Very Stiff Worm-Like Filomicelles can Still Enter Tumor Cells.

It is already well known that spherical micelles with a size of a few hundred nanometers can readily gain uptake by the tumor cells through endocytosis. However, considering the significantly increased length of filomicelles up to micron scale, and their established persistent length, it is intriguing to determine how and the extent to which the filomicelles are internalized by the cells. As a result, highly stiff (persistent length ˜10 μm) and extended (contour length ˜22 μm) filomicelles made of OS diblock polymer were used to determine whether the elevated stiffness would adversely affect or even abolish the ability of filomicelles to enter the tumor cells. OS filomicelles were selected because compared to them, OCL3 filomicelles should be more favored to enter cells since the persistent (˜5 μm) and contour lengths (˜7 μm) of OCL3 filomicelles are significantly lower than comparable sizes found in OS filomicelles.

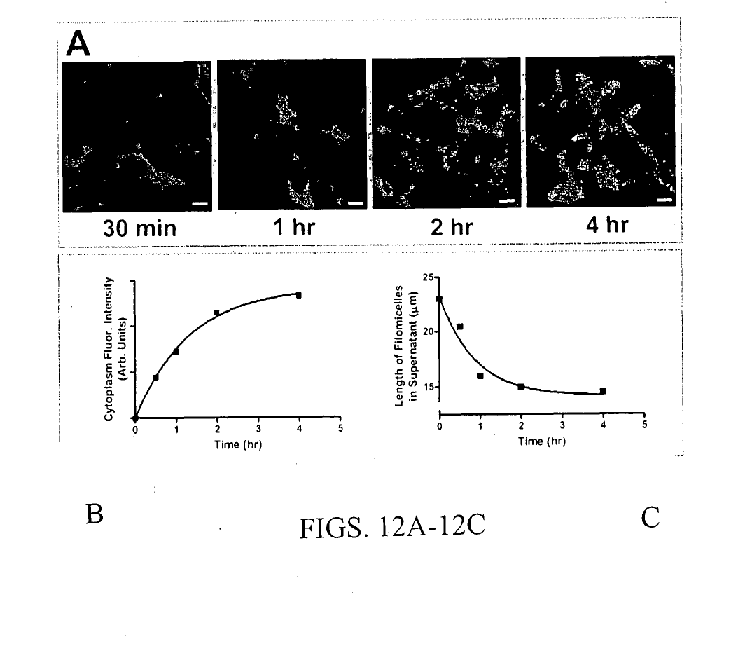

A highly lipophilic dye PKH26 was simultaneously used to label the hydrophobic core of the OS filomicelles. The interaction between PKH26 and the hydrophobic core was instant and strong enough so that the OS filomicelles could be labeled immediately and stably during the experiment. As seen in FIG. 12A, PKH26-labeled OS filomicelles started to accumulate on the A549 cell membrane within 60 minutes, then gradually transferred from the membrane into the cells. As the fluorescence intensity of the membrane decreased, small dots and aggregates could be seen inside the cells, resulting in increased intracellular fluorescence intensity (FIG. 12B). Conversely, the length of the OS filomicelles remaining in the media was also reduced (FIG. 12C). Accordingly, it appeared that the OS filomicelles fragmentized when they collided with the cell membrane, and thus their length became shortened. After this process, filomicelles having a length less than a certain level, seen to be about 14-15 μm in these experiments (FIG. 12C), may be quickly dissociated when they contact the cell surfaces by a “chewing up” process, instead of being released back into the media.

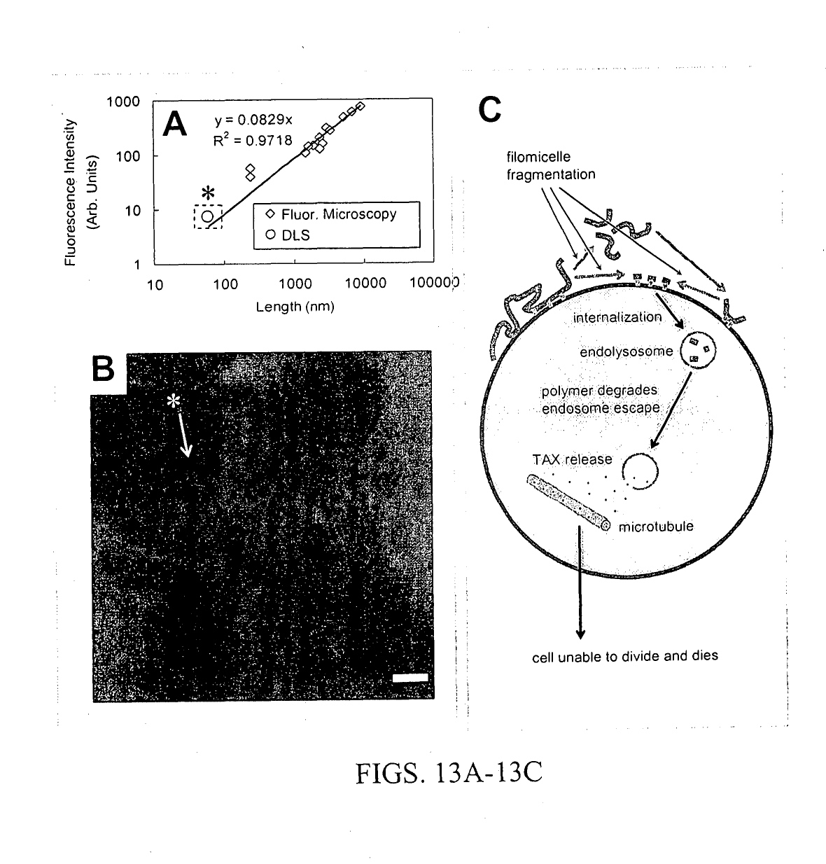

To further understand how the filomicelles enter the cells, a correlation between the size of the micelles and their fluorescence intensity was established (FIG. 13A). It is obvious that the fluorescence intensity is directly proportional to the length of the filomicelles, even when the length has been reduced to approximately sphere-size (260 nm, determined by dynamic light scattering (DLS) as described above, in an extreme case incorporated herein by reference. Thus, this correlation can be used to estimate the approximate size of the micelles when they are internalized into cells. As is shown in FIG. 13B, only small dots, not long fragments of the filomicelles, were seen inside the cells (indicated by the star). Therefore, it appears to be highly likely that the stiff worm-like filomicelles had to be dissociated into small nano-spheres before they could enter the cells through endocytosis (FIG. 13C). On the whole, it is clearly demonstrated that worm-like filomicelles, even with significantly extended length and fairly high stiffness, still enter the tumor cells to effectively deliver the drugs.

### Example 3

### Shape Affects the Delivery of Drugs Under Flow Conditions

While studies showed that filomicelles loaded with the anticancer drug paclitaxel will shrink tumors, with longer cylinders proving more effective at a given dose, providing effective filamentous carrier systems, the effects of shape in biological systems at the nanoscale were also evaluated. To test filomicelles directly as drug-delivery vehicles for cancer therapy, tumor-bearing nude mice (Ahmed et al., Mol. Pharmacol. 3:340-350 (2006)) were given a single tail-vein injection of either free drug or drug-loaded filomicelle. Saline and empty filomicelles served as control injections. The hydrophobic anticancer drug paclitaxel was injected at the maximum tolerated free drug dose of 1 mg kg⁻¹, or was loaded as recently described (Geng et al., Polymer 47:2519-2525 (2006)) at 1 or 8 mg kg⁻¹ into the hydrophobic cores of either 1 μm or 8 μm filomicelles. Higher doses of drug were not tried, as the intent in this experiment was a first comparison of shape and size effects.

Methods: Filomicelles were prepared from hydration of di-block copolymers with no residual co-solvent as previously described by Dalhaimer et al., supra 2003 and Geng et al., J Am Chem. Soc. 127:12780-12781 (2005)). Block copolymers of PEG-polyethylethylene (EOₘ-EEₙ, designated OE) or PEG-polycaprolactone (EOₘ-Clₙ, designated OCL) were synthesized by standard polymerizations by the method of Jain et al., Science 300:460-464 (2003). The diblocks used include OE7′, OCL1 and OCL3 as described above. Note that OE7′ is a copolymer related to those used in previous studies of vesicles, but the present copolymer has a slightly higher volume fraction of PEG (f_{EO}) that is more consistent with cylinder micelle formation, and also with cylinder micelles from the two OCL copolymers with similar f_{EO}. Cryo-transmission electron microscopy allowed visualization of the hydrophobic core of the micelles, and the total diameter dₒ is estimated to be about twice the core diameter.

Filomicelles were visualized by optical microscopy with a hydrophobic membrane marker, PKH26 (Sigma), which partitions into the cores of the filomicelles when added to a hydrated sample. Cell membrane probe, Fluorescein DHPE, nuclei Hoechst stain and Lysotracker Blue were from Molecular Probes.

Fluorescent and bright-field images were recorded using an Olympus IX71 inverted microscope with a CCD camera (Cascade 512, Roper Scientific). Repeated extrusion of filomicelle samples at 100-200 p.s.i. through a 400-nm membrane gently fragments the cylinders, which under these conditions led to a maximum contour length <10 μm, which can be controlled by the repetitions in extrusion.

For circulation studies, previous polymer vesicle protocols (as cited above from the Discher laboratory) were followed, and performance was assessed in two rodent species for comparison with previous findings. Male Sprague-Dawley rats were injected with 0.5 ml of 5 mg ml⁻¹ copolymer in phosphate buffered saline; alternatively, equal numbers of male or female C57 mice (with similar results) were injected with 0.1 ml of the same. Orbital bleeds into heparin tubes were taken at various times during the study. The plasma (containing the filomicelles) was separated from the other blood components by centrifugation at 7,000 g for 10 min to determine the number, N, and the contour lengths of the filomicelles in circulation. By comparison to non-centrifuged control samples, this level of centrifugation has no effect on measured length distributions and essentially separates into the supernatant plasma all of the micelles (or vesicles). Nₒ is the number of filomicelles from a one-hour bleed for the figures shown.

Additional circulation studies in four mice showed that (1) filomicelles in blood are statistically the same in number at time points of 1-2, 10, 30 and 60 min, and (2) the pre-injected concentration is statistically the same as that at 1-2 min when corrected for dilution into the typical blood volume of mice. Organs were retrieved and sectioned (5-μM slices) using a microtome. In each study using blood, citrate or EDTA was used as anticoagulant.

In vitro phagocytosis assays were performed on blood-drawn human neutrophils and also on a human macrophage cell line, THP1 (ATCC). Filomicelle suspensions of 0.1 mg copolymer (large excess for the number of cells) were incubated with the macrophage cell line for 24 h. In vitro assays of internalization of inert filomicelles by human lung-derived cells A549 (ATCC) were performed by incubation of cells pre-labelled with fluorescein-phosphatidylethanolamine (FL-DHPE, Molecular Probes) with filomicelles (red dye, PKH26) for preset times. This was followed by removing the supernatant of the remaining filomicelles and washing the cells with PBS three times before imaging. Subsequent fluorescent intensity analysis was used to quantify uptake. Nevertheless, uptake by macrophages depends on activation and is not just a passive adsorption process, e.g., omitting the PMA (phorbol-12-myristate-13-acetate) activation led to cells with control intensities.

Tumor studies were conducted in a similar way to those recently reported by Ahmed et al., Mol. Pharmacol. 3(3):340-350 (2006), but using the filomicelle delivery system, with A549 cells. Briefly, cultured cells were injected subcutaneously onto the backs of nude mice and allowed to grow until they reached a mean size of 0.52 cm² (±0.02 em²). Mice were then injected in the tail vein either with saline or filomicelle controls, paclitaxel in ethanol (1 mg kg⁻¹ which was the maximum tolerated dose for paclitaxel), or the same mass of worm-like filomicelles as above, except that the filomicelles were preloaded with paclitaxel as described by Geng et al, Polymer 47:2519-2525 (2006). Each group consisted of four mice. No group of mice showed any significant differences in weight change, and the maximum tolerated dose (MTD) was determined in separate studies to be the dose that causes 10% weight loss within 24 h (average from three mice), see Example 2 above. For paclitaxel-loaded filomicelles, the MTD exceeds the 8 mg kg⁻¹ used here by more than twofold. For unloaded OCL3 filomicelles (8 mm long), up to 300 mg kg⁻¹ of copolymer were injected without any significant weight loss in mice.

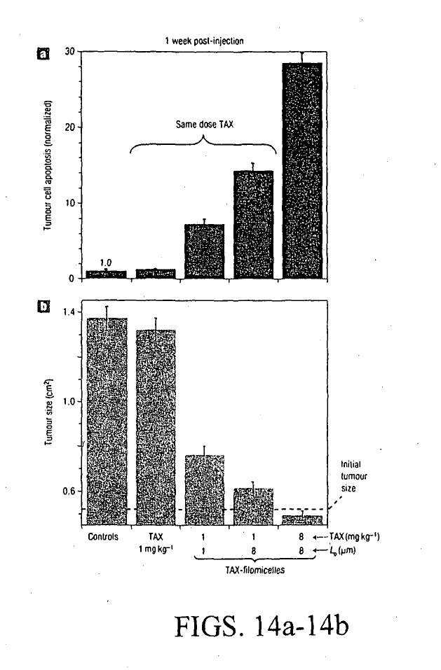

As shown in FIGS. 14A and 14B, filomicelles mediated paclitaxel (TAX) delivery to rapidly growing tumor xenografts on nude mice. As compared with controls using saline, empty OCL3 filomicelles, TAX as free drug in ethanol at MTD, or TAX loaded at two doses into the hydrophobic cores of filomicelles of two lengths, one week after administration apoptosis there was little effect seen for the free drug when measured by quantitative imaging of TUNEL-stained tumor sections—but quantifiably increasing cell death with increasing Lₒ was seen with increasing paclitaxel dosage (FIG. 14A).

Moreover, tumor size decreased directly with increasing apoptosis of the target cells, with tumor shrinkage evident for the longest filomicelles at the highest TAX dose (FIG. 14B), demonstrating clear advantages of the filomicelle as a paclitaxel carrier seven days post-injection. An eight-fold increase in filomicelle length for a 1 mg kg⁻¹ paclitaxel dosage was found to have about the same relative therapeutic effect as an eight-fold increase in the paclitaxel dosage—but without the added toxicity of the 8-fold increase in the drug load. Both increases lead to a doubling of the apoptosis that is measurable in the tumor, and both increases also lead to a similar relative decrease in tumor size. For comparison, phase I clinical trials with paclitaxel-loaded spherical micelles of PEG-(polylactic acid) used approximately an eight-fold higher paclitaxel dosage in each of three injections (Kim et al., Clin. Cancer Res. 10:3708-3716 (2004)).

In sum, an enhanced quantity of a drug, such as TAX, is distributed intracellularly in a tumor when delivered by the filomicelles nanocarriers, whereas a lowered dosage of the drug was shown to be delivered to the non-tumor organs, and as compared to delivery by spherical micelles. Thus, the novel filomicelle delivery system offers better tumor-selective biodistribution of a drug, and a reduced toxicity of the encapsulated drug to other organs. Consequently, special attention was paid to the animal maximum tolerated dose (MTD) of the drug that was delivered. The tumor selectivity of the cytotoxic effect and drug distribution was also evaluated, and the results demonstrated the advantages in MTD promotion and better tumor selection when the worm-like micelles were used for in vivo, delivery of the encapsulated drug, and establishes worm-like filomicelles as a novel system for drug delivery.

The disclosures of each patent, patent application and publication cited or described in this document are hereby incorporated herein by reference, in their entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

1. A stable, purely synthetic, self-assembling, controlled release, polyethylene oxide (PEO)-based block copolymer, filomicelle nanocarrier system for bio-selectively delivering a cytotoxic, anticancer therapeutic active agent to a cell, the system comprising:
filomicelles having a semi-permeable, thin-walled, amphiphilic, high molecular weight PEO-based block copolymer encapsulating membrane comprising a copolymer having a hydrophilic PEO component and at least one hydrolytically degradable, hydrophobic block component to effect controlled polyester chain hydrolysis in the membrane, such that when the at least one hydrophobic block component is combined with the hydrophilic PEO component, the PEO volume fraction (f_{EO}) and chain chemistry control encapsulant release kinetics from the filomicelle by membrane destabilization; and
a cytotoxic therapeutic active agent encapsulated therein to form at least one encapsulant;
wherein the PEO-based filomicelle membrane has a desired controlled release rate for bioselectively releasing the at least one encapsulant.

2. The system of claim 1, wherein the polyethylene oxide component of the copolymer comprises polyethylene glycol (PEG), or structural equivalent thereof.

3. The system claim 2, wherein the at least one hydrophobic block component comprises a hydrolytically degradable polyester.

4. The system of claim 1, further comprising increasing the mole fraction (mol %) of the at least one hydrolytically degradable, hydrophobic block component blended into the copolymer to directly control release of the at least one encapsulant upon subsequent hydration or fragmentation.

5. The system of claim 1, wherein increasing the block f_{EO} increases rate of transformation into a detergent-like moiety, thereby accelerating destabilization of bilayer morphology of the filomicelle membrane and encapsulant release.

6. The system of claim 1, wherein the at least one encapsulant comprises an amphiphilic or lipophilic cytotoxic composition.

7. The system of claim 1, wherein the at least one encapsulant comprises a hydrophilic cytotoxic active agent encapsulated in the lumen of the filomicelle, or the at least one encapsulant comprises a hydrophilic cytotoxic encapsulant encapsulated by intercalation into the filomicelle membrane, or there are one or more encapsulants selected from one or more hydrophilic encapsulants or one or more hydrophobic encapsulants, or a combination thereof.

8. The system of claim 7, wherein the hydrophilic cytotoxic encapsulant is selected from the group consisting of carbohydrates, including sucrose; marker-tagged dextrans, including fluorescent dextrans from 1 kD up to 200 kD; therapeutic compositions, including doxorubicin (DOX) or amphoterican B or paclitaxel (TAX); dyes; indicators; protein or protein fragments, salts; gene or gene fragments and oligonucleotides.

9. The system of claim 8, wherein the at least one therapeutic composition comprises an anti-cancer drug selected from cytotoxic doxorubicin and paclitaxel, or a combination thereof.

10. The system of claim 1, wherein the at least one encapsulant is encapsulated simultaneously with filomicelle formation, or subsequent thereto.

11. A method of bioselectively delivering a cytotoxic active agent to a cell from an active agent encapsulant-loaded, hydrolysis triggered, controlled release filomicelle delivery system produced by the system of claim 1, the method of delivery comprising:
selecting the at least one hydrolytically degradable, hydrophobic block component to effect controlled polyester chain hydrolysis in the membrane, such that when combined with the hydrophilic PEO component, the PEO volume fraction (f_{EO}) and chain chemistry control encapsulant release kinetics from the copolymer vesicles and filomicelle carrier membrane destabilization;
forming stable, purely synthetic, self-assembling, controlled release, PEO-based filomicelles, having a semi-permeable, thin-walled, amphiphilic, high molecular weight PEO-based block copolymer encapsulating membrane, and having a desired controlled release rate for releasing the cytotoxic therapeutic encapsulant; and
encapsulating therein the anticancer therapeutic active agent to form the at least one encapsulant; and delivering same to a cell.

12. The method of claim 11, wherein the at least one encapsulant comprises a hydrophilic cytotoxic active agent encapsulated in the lumen of the filomicelle, or the at least one encapsulant comprises a hydrophilic cytotoxic encapsulant encapsulated by intercalation into the filomicelle membrane, or there are one or more encapsulants selected from one or more hydrophilic encapsulants or one or more hydrophobic encapsulants, or a combination thereof.

13. The method of claim 12, wherein at least one hydrophilic cytotoxic encapsulant is selected from the group consisting of carbohydrates, including sucrose; marker-tagged dextrans, including fluorescent dextrans from 1 kD up to 200 kD; therapeutic compositions, including doxorubicin (DOX) or amphoterican B or paclitaxel (TAX); dyes; indicators; protein or protein fragments, salts; gene or gene fragments and oligonucleotides.

14. The method of claim 13, wherein the therapeutic composition comprises an anti-cancer drug selected from cytotoxic doxorubicin and paclitaxel, or a combination thereof.

15. The method of claim 11, wherein the at least one encapsulant is encapsulated simultaneously with filomicelle formation, or subsequent thereto.

16. A method of releasing the at least one encapsulant from the loaded, hydrolysis triggered, controlled release filomicelle prepared by the method of claim 11, to a cellular target in vitro or in vivo, wherein the method comprises:
delivering the filomicelle and the at least one encapsulant contained therein to an intended cellular target, wherein the composition of the cellular target environment triggers hydrolysis or fragmentation at a predetermined rate of the filomicelle membranes; and
thereby effecting release of the at least one encapsulant into the target cell.

17. The method of claim 16, wherein the delivering further comprises administering the filomicelle to a patient in need thereof, and further comprising releasing the at least one encapsulant from the filomicelle to the patient, wherein the filomicelle and at least one encapsulant are biocompatible.

18. The method of claim 17, wherein the at least one encapsulant comprises more than one cytotoxic composition, acting in combination.

19. The method of claim 18, wherein following releasing the at least one encapsulant in the cells of the patient, the method further comprises effecting quantifiable shrinkage of solid tumors.

20. The method of claim 19, wherein following releasing the at least one encapsulant in the cells of the patient, the method further comprises effecting quantifiable apoptosis of tumor cells within 1-2 days post delivery.

21. The method of claim 17, wherein the at least one encapsulant comprises a hydrophilic cytotoxic active agent encapsulated in the lumen of the filomicelle, or the at least one encapsulant comprises a hydrophilic cytotoxic encapsulant encapsulated by intercalation into the filomicelle membrane, or there are one or more encapsulants selected from one or more hydrophilic encapsulants or one or more hydrophobic encapsulants, or a combination thereof.

22. The method of claim 21, wherein the hydrophilic cytotoxic encapsulant is selected from the group consisting of carbohydrates, including sucrose; marker-tagged dextrans, including fluorescent dextrans from 1 kD up to 200 kD; therapeutic compositions, including doxorubicin (DOX) or amphoterican B or paclitaxel (TAX); dyes; indicators; protein or protein fragments, salts; gene or gene fragments and oligonucleotides.

23. The method of claim 22, wherein the therapeutic composition comprises an anti-cancer drug selected from cytotoxic doxorubicin and paclitaxel, or a combination thereof.

24. The method of claim 16, wherein the at least one encapsulant is encapsulated simultaneously with filomicelle formation, or subsequent thereto.
